SEQUENCE LISTING (8 sequences)

SEQ ID NO: 1 - protein, 8 aa, Artificial Sequence
  Description of Artificial Sequence Synthetic peptide
DYKDDDDK

SEQ ID NO: 2 - protein, 23 aa, Artificial Sequence
  Description of Artificial Sequence Synthetic peptide
KRITVEEALAHPYLEQYYDPTDE

SEQ ID NO: 3 - protein, 6 aa, Artificial Sequence
  Description of Artificial Sequence Synthetic 6xHis tag
HHHHHH

SEQ ID NO: 4 - protein, 10 aa, Artificial Sequence
  Description of Artificial Sequence Synthetic peptide
EQKLISEEDL

SEQ ID NO: 5 - protein, 9 aa, Artificial Sequence
  Description of Artificial Sequence Synthetic peptide
YPYDVPDYA

SEQ ID NO: 6 - protein, 11 aa, Artificial Sequence
  Description of Artificial Sequence Synthetic peptide
YTDIEMNRLGK

SEQ ID NO: 7 - protein, 11 aa, Artificial Sequence
  Description of Artificial Sequence Synthetic peptide
QPELAPEDPED

SEQ ID NO: 8 - protein, 9 aa, Artificial Sequence
  Description of Artificial Sequence Synthetic peptide
CDYKDDDDK

(19) United States
(12) Patent Application Publication
(10) Pub. No.: US 2017/0097339 A1
(43) Pub. Date: Apr. 6, 2017

(54) Analyte Detection with Multiple Substrates

(71) Applicant: TGR Biosciences Pty Ltd., Thebarton (AU)

(72) Inventors: Antony James Sheehan, Seaview Downs (AU); Michael Francis Crouch, Hackney (AU)

(21) Appl. No.: 15/282,634

(22) Filed: Sep. 30, 2016

Related U.S. Application Data
(60) Provisional application No. 62/236,676, filed on Oct. 2, 2015.
(60) Provisional application No. 62/237,522, filed on Oct. 5, 2015.

(51) Int. Cl. G01N 33/543

(52) U.S. Cl. CPC G01N 33/54306

(57) ABSTRACT

The present disclosure provides methods and/or kits for detecting an analyte in a sample. Some embodiments provide an analyte detection system, kit, and method of using the same, comprising: a first agent comprising at least one tag and capable of binding to the analyte; a second agent covalently bound to a plurality of peptide tags and capable of binding to the analyte; a first bead comprising a binder capable of binding with the tagged first agent; and a second bead comprising an anti-peptide agent capable of binding with at least one of the plurality of covalently bound peptide tags.

### CROSS REFERENCE

This application claims the benefit of priority from both U.S. Provisional Patent Application No. 62/236,676, filed Oct. 2, 2015, and U.S. Provisional Patent Application No. 62/237,522, filed Oct. 5, 2015. All of the foregoing related applications, in their entirety, are incorporated herein by reference.

### FIELD OF THE DISCLOSURE

The present disclosure relates to methods for detecting an analyte in a sample.

### SEQUENCE LISTING

The instant application contains a Sequence Listing which has been submitted electronically in ASCII format and is hereby incorporated by reference in its entirety. Said ASCII copy, created on Sep. 27, 2016, is named 013018-0008-999_SL.txt and is 2,227 bytes in size.

### BACKGROUND

Detection of analytes in samples is important in many industries including, for example, research, immunology, water quality assessment, environmental science and engineering, medicine, etc. See, for example, U.S. Pat. No. 9,086,407 and U.S. patent application Ser. No. 15/222,376, which are incorporated herein by reference in their entirety.

Different methods for detecting analytes in samples may be used, including, for example, high pressure liquid chromatography (HPLC), mass spectrometry, and enzyme linked immunosorbent assays (ELISA). While HPLC and mass spectrometry may be used to detect analytes on the basis of charge and/or size, ELISA may be used to detect an analyte based on antigens on the analyte that are recognizable by capture and detection agents (e.g. antibodies, aptamers, etc.), making it an important assay, especially in the life sciences. ELISA may be used to detect the presence, absence or the amount of an analyte in a sample.

While conventional ELISA is a widely used method, there is a continuing need to develop improved assays with lower cost and reduced assay time. First, conventional ELISA can require expensive capture and detection agents, such as antibodies. Thus, techniques with reduced antibody requirements would decrease expense of assays. In addition, conventional ELISA takes at least 2 hours to complete and generally includes at least 2 separate incubation and washing steps. Accordingly, it would be desirable to provide a method for detecting an analyte in a sample that takes less time and inputs to perform compared with conventional ELISA, while maintaining or improving the sensitivity of detection.

Reference to any prior art in this specification is not, and should not be taken as, an acknowledgment or any form of suggestion that this prior art forms part of the common general knowledge in any country.

### SUMMARY

Certain embodiments may provide a system for detecting an analyte, the system comprising: a first agent (for example, a monoclonal antibody, a polyclonal antibody, a multivalent antibody, a chimeric antibody, a multispecific antibody, or an antibody fragment thereof; an aptamer; an affimer; a protein; a protein receptor; a protein receptor; a protein ligand; or a fusion protein, comprising, for example, an immunoglobulin fusion partner, a fusion partner that stabilizes a receptor or a ligand, or a fusion partner that provides a target for binding) comprising at least one tag (for example a biotin or peptide tag) and capable of binding to the analyte, a second agent (for example, a monoclonal antibody, a polyclonal antibody, a multivalent antibody, a chimeric antibody, a multispecific antibody, or an antibody fragment thereof; an aptamer; an affimer; a protein; a protein receptor; a protein receptor; a protein ligand; or a fusion protein, comprising, for example, an immunoglobulin fusion partner, a fusion partner that stabilizes a receptor or a ligand, or a fusion partner that provides a target for binding) covalently bound to a plurality of peptide tags and capable of binding to the analyte, a first bead comprising a binder (for example, a monoclonal antibody, a polyclonal antibody, a multivalent antibody, a chimeric antibody, a multispecific antibody, or an antibody fragment thereof; an aptamer; an affimer; a protein; a protein receptor; a protein receptor; a protein ligand; a fusion protein, comprising, for example, an immunoglobulin fusion partner, a fusion partner that stabilizes a receptor or a ligand, or a fusion partner that provides a target for binding; avidin, streptavidin, an avidin derivative, or a streptavidin derivative) capable of binding with the tagged first agent, and a second bead comprising an anti-peptide agent (for example, a monoclonal antibody, a polyclonal antibody, a multivalent antibody, a chimeric antibody, a multispecific antibody, or an antibody fragment thereof; an aptamer; an affimer; a protein; a protein receptor; a protein receptor; a protein ligand; or a fusion protein, comprising, for example, an immunoglobulin fusion partner, a fusion partner that stabilizes a receptor or a ligand, or a fusion partner that provides a target for binding) capable of binding with at least one of the plurality of covalently bound peptide tags.

Certain embodiments may provide a system for detecting an analyte, the system comprising: a plurality of a first agent capable of binding to the analyte and a plurality of a second agent capable of binding to the analyte. Each of the first agent may comprise: a first region capable of binding with a first antigenic aspect of the analyte, and a plurality of first tags. Each of the second agent may comprise: a second region capable of binding with a second antigenic aspect of the analyte, and a plurality of covalently bound peptide second tags. The system may further comprise a first bead and a second bead. The first bead (for example a latex bead; for example a bead coated or containing a substance configured to emit singlet oxygen upon stimulation at one or more wavelengths of light) may comprise a plurality of binders conjugated thereon, wherein at least one of the plurality of binders is capable of selectively binding with at least one of the plurality of first tags. The second bead (for example a latex bead; for example a bead containing or coated with a Europium or Terbium compound configured to emit light at a pre-determined wavelength after activation with singlet oxygen followed by irradiation) may comprise a plurality of anti-peptide agents conjugated thereon, wherein at least one of the anti-peptide agents is capable of selectively binding with a least one of the plurality of covalently bound peptide second tags. The first bead and the second bead may be capable of detectably interacting (for example by chemical transfer interaction) when the first bead and the second bead are disposed within a certain distance of one another.

Certain embodiments may provide a system for detecting at least two analytes, comprising: a first donor agent capable of binding to a first analyte; a first binder conjugated to a donor bead, said first binder capable of binding to the first donor agent; a tagged second donor agent capable of binding to a second analyte; a second binder conjugated to the donor bead, said second binder capable of binding to the tagged second donor agent; a tagged first acceptor agent capable of binding to the first analyte, the tagged first acceptor agent comprising a plurality of covalently bound first peptide tags; and a first acceptor bead conjugated to an anti-first peptide agent, the anti-first peptide agent capable of binding with at least one of the plurality of covalently bound peptide tags; a second acceptor agent capable of binding with the second analyte; and a second acceptor bead capable of binding with the second acceptor agent. In certain embodiments, the binder and the second binder may be the same type of binder. In certain embodiments, the second acceptor agent may be directly conjugated to the second acceptor bead. In certain embodiments, the second acceptor agent may be a tagged second acceptor agent comprising a plurality of covalently bound second peptide tags.

Certain embodiments may provide an analyte detection kit, comprising: a first agent comprising at least one tag and capable of binding to the analyte, a second agent covalently bound to a plurality of peptide tags and capable of binding to the analyte, a first bead comprising a binder capable of binding with the tagged first agent, and a second bead comprising an anti-peptide agent capable of binding with at least one of the plurality of covalently bound peptide tags. In certain embodiments, an analyte detection kit may have a storage life of at least 9 months, such as at least 1 year, or at least 2 years.

Certain embodiments may provide an analyte detection kit, comprising: a first agent comprising at least one tag and capable of binding to the analyte, a second agent covalently bound to a plurality of peptide tags and capable of binding to the analyte, a donor bead comprising a binder capable of binding with the tagged first agent, and an acceptor bead comprising an anti-peptide agent capable of binding with at least one of the plurality of covalently bound peptide tags.

Certain embodiments may provide a series of complementary analyte detection kits that may be used to detect a plurality of analytes in a single sample, comprising at least a first kit and a second kit. The first kit may comprise: a first agent comprising at least one tag and capable of binding to a first analyte of the plurality of analytes, a second agent covalently bound to a plurality of peptide tags and capable of binding to the first analyte, a donor bead comprising a binder capable of binding with the tagged first agent of the first kit, and an acceptor bead comprising an anti-peptide agent capable of binding with at least one of the plurality of covalently bound peptide tags present on the second agent of the first kit. The second kit may comprise: a first agent comprising at least one tag and capable of binding to a second analyte of the plurality of analytes, a second agent covalently bound to a plurality of peptide tags and capable of binding to the second analyte, a donor bead comprising a binder capable of binding with the tagged first agent of the second kit, and an acceptor bead comprising an anti-peptide agent capable of binding with at least one of the plurality of covalently bound peptide tags present on the second agent of the second kit. In certain embodiments, a common binder may be used in the first kit and the second kit. In certain embodiments, a common donor bead may be used in the first kit and the second kit. In certain embodiments, a common peptide tag and a common anti-peptide agent may be used in the first kit and the second kit. In certain embodiments, a common acceptor bead may be used in the first kit and the second kit.

Certain embodiments may provide a series of complementary analyte detection kits that may be used to detect a plurality of analytes within the same sample, comprising at least a first kit and a second kit. The first kit may comprise: a first agent comprising at least one tag and capable of binding to a first analyte of the plurality of analytes, a second agent covalently bound to a plurality of peptide tags and capable of binding to the first analyte, a donor bead comprising a binder capable of binding with the tagged first agent of the first kit, and an acceptor bead comprising an anti-peptide agent capable of binding with at least one of the plurality of covalently bound peptide tags present on the second agent of the first kit. The second kit may comprise: a first agent comprising at least one tag and capable of binding to a second analyte of the plurality of analytes, a second agent covalently bound to a plurality of peptide tags and capable of binding to the second analyte, a donor bead comprising a binder capable of binding with the tagged first agent of the second kit, and an acceptor bead comprising an anti-peptide agent capable of binding with at least one of the plurality of covalently bound peptide tags present on the second agent of the second kit. In the exemplary embodiments providing the series of complementary analyte detection kits, the analyte detection signal from the first kit may be different from the analyte detection signal from the second kit.

Certain embodiments may provide a multi-analyte detection kit, comprising: a first agent comprising at least one tag and capable of binding to a first analyte, a second agent covalently bound to a plurality of peptide tags and capable of binding to the first analyte, a donor bead comprising a binder capable of binding with the tagged first agent, an acceptor bead comprising an anti-peptide agent capable of binding with at least one of the plurality of covalently bound peptide tags present on the second agent, a third agent comprising at least one tag and capable of binding to a second analyte, a fourth agent covalently bound to a plurality of peptide tags and capable of binding to the second analyte; a further donor bead comprising a binder capable of binding with the tagged third agent, and a further acceptor bead comprising an anti-peptide agent capable of binding with at least one of the plurality of covalently bound peptide tags present on the fourth agent. In certain embodiments, the donor bead and the further donor bead are the same bead.

Certain embodiments may provide a multi-analyte detection kit, comprising: a first agent comprising at least one tag and capable of binding to a first analyte, a second agent covalently bound to a plurality of peptide tags and capable of binding to the first analyte; a donor bead comprising a binder capable of binding with the tagged first agent, and an acceptor bead comprising an anti-peptide agent capable of binding with at least one of the plurality of covalently bound peptide tags present on the second agent, a third agent comprising at least one tag and capable of binding to a second analyte, a fourth agent covalently bound to a plurality of peptide tags and capable of binding to the second analyte, a further donor bead comprising a binder capable of binding with the tagged third agent, and a further acceptor bead comprising an anti-peptide agent capable of binding with at least one of the plurality of covalently bound peptide tags present on the fourth agent. The acceptor bead and the further acceptor bead may emit different signals.

Certain embodiments may provide an analyte detection kit, comprising: a first bead comprising a binder capable of binding with a tagged first agent, a plurality of peptide tags capable of being covalently bound to a second agent, wherein the second agent is capable of binding to a specific analyte, and a second bead comprising an anti-peptide agent capable of binding with at least one of the plurality of peptide tags capable of being covalently bound to a second agent.

Certain embodiments may provide a method for detecting an analyte, comprising: binding an analyte with a first agent and a second agent. The first agent may comprise one or more first tags. The second agent may comprise a plurality of covalently bound peptide second tags. The method may further comprise: attaching at least one of the one or more first tags to a binder conjugated to a first bead, binding at least one of the plurality of covalently bound peptide second tags to an anti-peptide agent conjugated to a second bead, and detecting the presence of the analyte by sensing an emission after an interaction between the attached first bead and the bound second bead. In certain embodiments, the method does not require any washing steps.

Certain embodiments may provide a method for detecting an analyte, comprising: binding a tagged first agent to the analyte and separately binding a tagged second agent to the analyte. The tagged first agent may comprise one or more first tags. The tagged second agent may comprise a plurality of covalently bound peptide second tags. The method may further comprise attaching at least one of the one or more first tags to a binder conjugated to a first bead, binding at least one of the plurality of covalently bound peptide second tags to an anti-peptide agent conjugated to a second bead, and detecting the presence of the analyte by sensing an emission resulting from an interaction between the attached first bead and the bound second bead.

Certain embodiments may provide a method for detecting an analyte, comprising: binding a first agent to the analyte and separately binding a tagged second agent to the analyte. The tagged second agent may comprise a plurality of covalently bound peptide tags. The method may further comprise attaching the first agent to a binder conjugated to a first bead, binding at least one of the plurality of covalently bound peptide tags to an anti-peptide agent conjugated to a second bead, and detecting the presence of the analyte by sensing an emission from the bound second bead resulting from an interaction between the attached first bead and said bound second bead.

Certain embodiments may provide a method for detecting an analyte, comprising: forming an analyte complex, wherein the analyte complex comprises: a first agent comprising one or more first tags and bound to the analyte; and a second agent covalently bound to a plurality of peptide tags and bound to said analyte. The method may further comprise: attaching at least one of the one or more first tags to a binder conjugated to a first bead, binding at least one of the plurality of covalently bound peptide second tags to an anti-peptide agent conjugated to a second bead, and detecting the presence of the analyte by sensing an emission resulting from an interaction between the attached first bead and the bound second bead.

Certain embodiments may provide a method for detecting the presence of, or determining the absence of, a specific analyte in a sample, comprising; mixing a tagged first agent and a tagged second agent into the sample. The tagged first agent may be capable of binding with the specific analyte. The tagged second agent may comprise a plurality of covalently bound peptide second tags and may be capable of separately binding with the specific analyte. The method may further comprise: capturing the tagged first agent with a first bead, binding the tagged second agent to a second bead, and detecting the presence of, or determining the absence of, the specific analyte by sensing an emission resulting from an interaction between the captured first bead and said bound second bead.

Certain embodiments may provide a method of detecting an analyte in the presence of a plurality of non-analyte antibodies, comprising: mixing a first agent and a tagged second agent into a sample comprising the analyte and the plurality of non-analyte antibodies. The tagged second agent may comprise a plurality of covalently bound peptide second tags. The method may further comprise: binding the first agent directly to the analyte and binding the tagged second agent directly to the analyte, and introducing a first bead comprising a binder and a second bead comprising an anti-peptide agent to the bound analyte. The binder may preferentially bind to the first agent over the non-analyte antibodies. The anti-peptide agent may be capable of binding to at least one of the covalently bound peptide second tags. The method may further comprise: detecting the presence of the analyte by sensing an emission resulting from an interaction between the attached first bead and the bound second bead.

Certain embodiments may provide a method of detecting an analyte in the presence of a plurality of non-analyte antibodies, comprising: mixing a first agent and a tagged second agent into a sample comprising the analyte and the plurality of non-analyte antibodies. The tagged second agent may comprise a plurality of covalently bound peptide second tags. The method may further comprise: binding the first agent directly to the analyte and binding the tagged second agent directly to the analyte, and introducing a first bead comprising a binder and a second bead comprising an anti-peptide agent to the bound analyte. The binder may preferentially bind to the first agent over the non-analyte antibodies. The anti-peptide agent may be capable of binding to at least one of the covalently bound peptide second tags. The method may further comprise: capturing the tagged first agent with the binder and binding at least one of the plurality of covalently bound peptide second tags to the anti-peptide agent conjugated to the second bead, and detecting the presence of the analyte by sensing an emission resulting from an interaction between the attached first bead and the bound second bead.

Certain embodiments may provide a method for detecting at least two different analytes in a sample, comprising: binding a first donor agent to a first analyte of the at least two analytes and separately binding a tagged first acceptor agent to the first analyte. The tagged first acceptor agent may comprise a plurality of covalently bound peptide tags. In certain further embodiments, the method may further comprise: binding a tagged second donor agent to a second analyte of the at least two analytes and separately binding a second acceptor agent to the second analyte, attaching the first donor agent to a first binder conjugated on a donor bead and attaching the second donor agent to a second binder conjugated on the donor bead, binding at least one of the plurality of covalently bound peptide tags to an anti-peptide agent conjugated to a first acceptor bead, binding the second acceptor agent to a second acceptor bead, and detecting the at least two different analytes by sensing the product of an interaction between the donor bead and the first and second acceptor beads. In certain embodiments, the method may comprise: binding a tagged second donor agent to a second analyte of the at least two analytes and separately binding a second acceptor agent to the second analyte, wherein the second acceptor agent is directly conjugated to a second acceptor bead, attaching the first donor agent to a first binder conjugated on a donor bead and attaching the second donor agent to a second binder conjugated on the donor bead, binding at least one of the plurality of covalently bound peptide tags to an anti-peptide agent conjugated to a first acceptor bead, and detecting the at least two different analytes by sensing the product of an interaction between the donor bead and the first and second acceptor beads.

Certain embodiments may provide a method for detecting at least two epitopes of an analyte, comprising: binding a tagged donor agent to the analyte, attaching the tagged donor agent to a binder conjugated on a donor bead, and binding a tagged first acceptor agent to a first epitope of the analyte and separately binding a second acceptor agent to a second epitope of the analyte. The tagged first acceptor agent may comprise a plurality of covalently bound peptide first tags. In certain further embodiments, the second acceptor agent may be bound to a second acceptor bead. In certain embodiments, the method may further comprise: binding at least one of the plurality of covalently bound peptide tags to an anti-peptide first agent conjugated to a first acceptor bead. In certain embodiments, the method may further comprise: binding at least one of the plurality of covalently bound peptide tags to an anti-peptide first agent conjugated to a first acceptor bead, binding the second acceptor agent to a second acceptor bead, and detecting the at least two epitopes of the analyte by sensing the product of an interaction between the donor bead and the two acceptor beads.

Certain embodiments may provide a detection complex, comprising: one or more analyte complexes, each comprising: an analyte, a first agent comprising one or more first tags and bound to the analyte, and a second agent covalently bound to a plurality of peptide tags and separately bound to said analyte. The detection complex may further comprise: a first bead having a plurality of binders conjugated thereon, wherein at least one of the one or more first tags present from the one or more analyte complexes is bound to one of the plurality of binders. The detection complex may further comprise: at least a second bead having a plurality of anti-peptide agents conjugated thereon, wherein at least one of the plurality of covalently bound peptide second tags from the one or more analyte complexes is bound to one of the plurality of anti-peptide agents. In certain embodiments, the at least second bead may be a plurality of beads. In certain embodiments, detection complex may comprise the first bead, a plurality of analyte complexes bound to the first bead, and each of the plurality of analyte complexes bound to a separate second bead.

Certain embodiments may provide a multi-analyte detection complex, comprising a first analyte complex and a second analyte complex. The first analyte complex may comprise: a first analyte; a tagged donor agent bound to the first analyte, the tagged donor agent comprising one or more donor tags; and a tagged acceptor agent bound to the first analyte, the tagged acceptor agent comprising a plurality of covalently bound peptide tags. The second analyte complex may comprise: a second analyte, a further donor agent bound to the second analyte, and a further acceptor agent bound to the second analyte. The multi-analyte detection complex may further comprise: a donor bead having a plurality of binders conjugated thereon, at least one of the one or more first tags present from each of the first and second analyte complexes bound to one of the plurality of binders, and a further donor agent bound to one of the plurality of binders. The multi-analyte detection complex may further comprise: a first acceptor bead having a plurality of anti-peptide agents conjugated thereon, at least one of the plurality of covalently bound peptide second tags bound to one of the plurality of anti-peptide agents. The multi-analyte detection complex may further comprise: a second acceptor bead bound to the further acceptor agent. In certain embodiments, the first acceptor bead comprises europium. In certain embodiments, the second acceptor bead comprises terbium. In certain embodiments, the multi-analyte detection complex further comprises a third acceptor bead and a fourth acceptor bead. In certain embodiments, the third acceptor bead comprises rubrene and the fourth acceptor bead comprises samarium.

Certain embodiments may provide a multi-epitope detection complex, comprising: an analyte complex, comprising: an analyte; a tagged donor agent bound to the analyte, the tagged donor agent comprising one or more donor tags; a tagged acceptor agent bound to a first epitope of the analyte, the tagged acceptor agent comprising a plurality of covalently bound peptide tags; and a further acceptor agent bound to a second epitope of the analyte. The multi-epitope detection complex may further comprise: a donor bead having a plurality of binders conjugated thereon, wherein at least one of the one or more donor tags is bound to one of the plurality of binders. The multi-epitope detection complex may further comprise: a first acceptor bead having a plurality of anti-peptide agents conjugated thereon, at least one of the plurality of covalently bound peptide second tags bound to one of the plurality of anti-peptide agents. The multi-epitope detection complex may further comprise: a second acceptor bead bound to the further acceptor agent.

Certain embodiments may employ methods for detecting, for example determining or quantifying, an analyte in a sample suspected of containing the analyte. One method may comprise treating a sample suspected of containing an analyte under conditions such that the analyte, if present, causes a photosensitizer (for example, a photosensitizer on a first bead or a donor bead), and a photoactive indicator precursor molecule (for example, a photoactive indicator precursor molecule present on a second bead or an acceptor bead) to come into close proximity. The photosensitizer generates singlet oxygen which activates the photoactive indicator precursor to generate a photoactive indicator molecule. Upon irradiation with light the photoactive indicator molecule produces light, which may be measured. The amount of light produced by the photoactive indicator is related to the amount of analyte in the sample. Compositions, kits, and compounds are also disclosed.

Certain embodiments may employ a method for detecting an analyte which is capable of directly or indirectly binding with a photosensitizer and/or a photoactive indicator precursor. In one embodiment, the method comprises a first step of providing in combination a sample suspected of containing an analyte; a photosensitizer capable in its excited state of generating singlet oxygen, wherein the photosensitizer is associated with the analyte; and a photoactive indicator precursor capable of forming a photoactive indicator upon reaction with singlet oxygen, wherein the photoactive indicator precursor is associated the analyte; then a second step of exciting the photosensitizer by irradiation with light; and a final step of measuring the fluorescence of the photoactive indicator. At least one of the photosensitizer and photoactive indicator precursor is capable of binding directly or indirectly to the analyte or to an agent complementary to the analyte. The fluorescence measured is related to the amount of the analyte in the sample. In another embodiment, the method comprises the first step of combining in an aqueous sample a sample suspected of containing an analyte; a first suspendible particle, for example a bead or a donor bead, comprised of a photosensitizer capable in its excited state of generating singlet oxygen, wherein the particle has a binder bound thereto; and a second suspendible particle, for example a bead or an acceptor bead, comprised of a photoactive indicator precursor capable of forming a photoactive indicator upon reaction with singlet oxygen, wherein the particle has a second binder, for example an anti-peptide agent or streptavidin, bound thereto; a second step of irradiating the sample to excite the photosensitizer to generate singlet oxygen; and a final step of measuring the fluorescence of the photoactive indicator. Each binder is capable of binding directly or indirectly with the analyte or to an agent, for example a tagged agent, complementary to the analyte. The fluorescence measured is related to the amount of the analyte in the sample.

In another embodiment, the method may comprise a first step of providing in combination a sample suspected of containing an analyte; a photosensitizer capable in its excited state of generating singlet oxygen, wherein the photosensitizer is associated with binder; and a suspendible particle, for example a bead or an acceptor bead, having bound thereto further binder, for example an anti-peptide agent, wherein the suspendible particle comprises a photoactive indicator precursor capable of forming a photoactive indicator upon reaction with singlet oxygen; a second step of irradiating the combination with light to excite the photosensitizer; and a final step of measuring the fluorescence of the photoactive indicator. Each binder is capable of binding directly or indirectly to the analyte or to an agent, for example a tagged agent, complementary to the analyte. The fluorescence measured is related to the amount of the analyte in the sample.

Certain embodiments may provide a method for detecting an analyte. The method may comprise a first step of providing in combination a sample suspected of containing an analyte; a photosensitizer capable in its excited state of generating singlet oxygen, wherein the photosensitizer is associated with a first binder; and a photoactive indicator precursor capable of forming a photoactive indicator upon reaction with singlet oxygen, wherein the photoactive indicator precursor is associated with a second binder; a second step of irradiating the combination with light to excite the photosensitizer; and a final step of measuring the fluorescence of the photoactive indicator. Each binder is capable of binding directly or indirectly to the analyte or to an agent complementary to the analyte. The fluorescence measured is related to the amount of the analyte in the sample.

In another embodiment, the analyte detection system may comprise 2 or more (for example 3 or more, or 4 or more) different types of acceptor beads each employing a photoactive indicator precursor that fluoresces at a different (or detectably different) wavelength. Each of the different types of acceptor beads may provide a different photoactive indicator. The different type of acceptor beads may comprise for example rubrene, europium (for example a europium chelate), samarium, or terbium, capable of reacting with singlet oxygen to produce a photoactive indicator capable of fluorescing.

Certain embodiments may provide a method for reducing inventory requirements necessary to make a plurality of multi-bead assay kits capable of detecting different analytes, comprising: inserting a universal acceptor bead capable of excitation via a chemical transfer interaction, the universal acceptor bead comprising a conjugated anti-peptide agent, the anti-peptide agent capable of binding with a certain type or amino acid sequence of peptide tag, into each of the plurality of assay kits.

In certain of the above embodiments, the anti-peptide second agent may bind to the donor (or photosensitive) bead while the tagged first agent is bound to the acceptor (or the photoactive indicator precursor) bead.

### DETAILED DESCRIPTION OF THE FIGURES

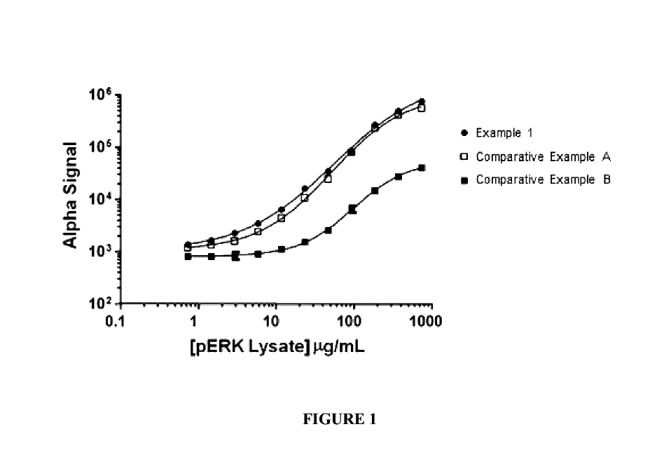

FIG. 1: A graph of the Alpha Signal vs. pERK Lysate Concentration for Example 1 and Comparative Examples A&B.

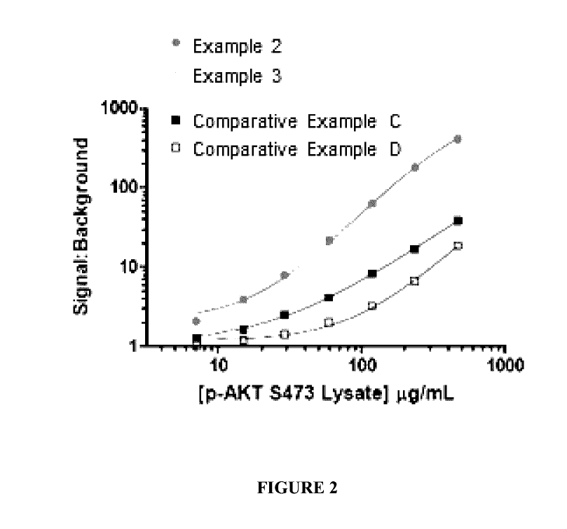

FIG. 2: A graph of the Signal to Background ratio vs. p-AKT 5473 Lysate concentration for Examples 2-3 and Comparative Examples C&D.

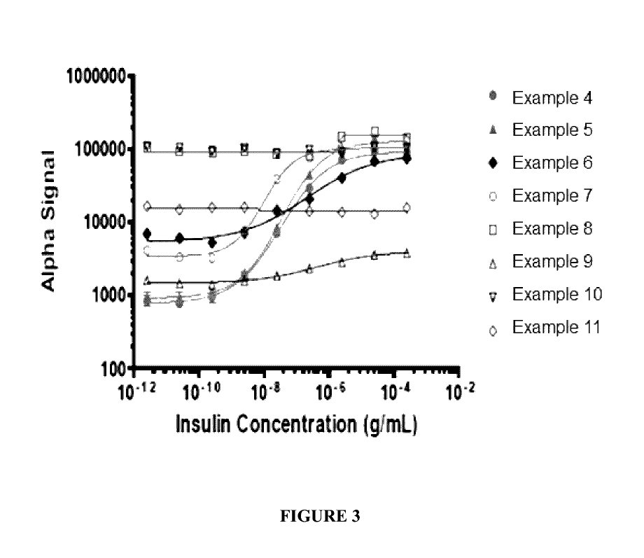

FIG. 3: A graph of the Alpha Signal vs. Insulin Concentration for Examples 4-11.

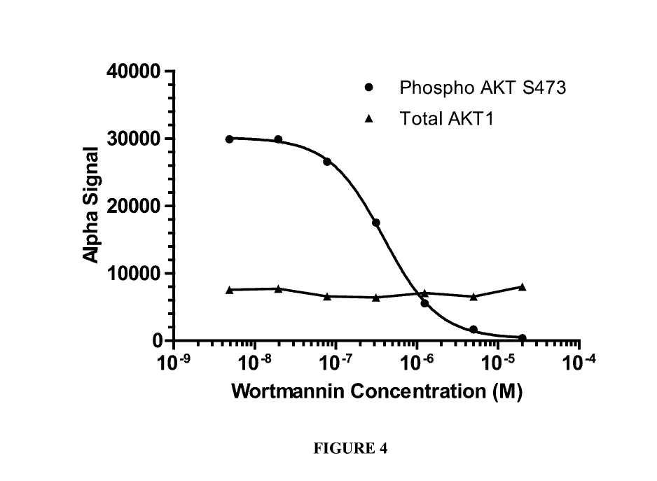

FIG. 4. A graph of Alpha Signal vs. Wortmannin Concentration for Example 12.

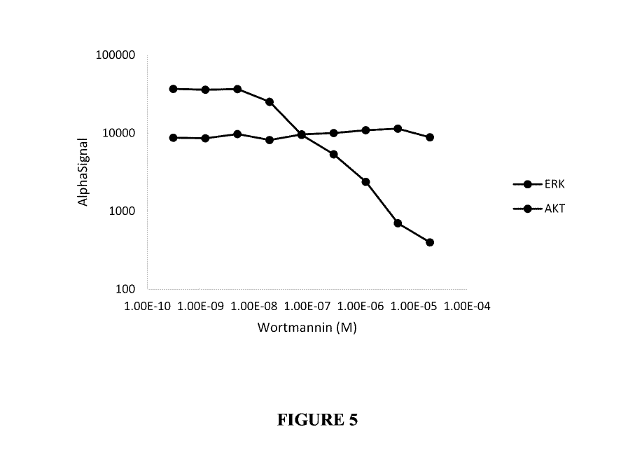

FIG. 5. A graph of Alpha Signal vs. Wortmannin Concentration for Example 13.

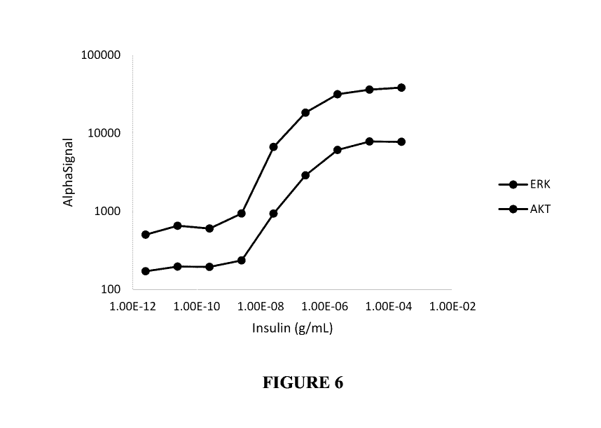

FIG. 6. A graph of Alpha Signal vs. Insulin Concentration for Example 14.

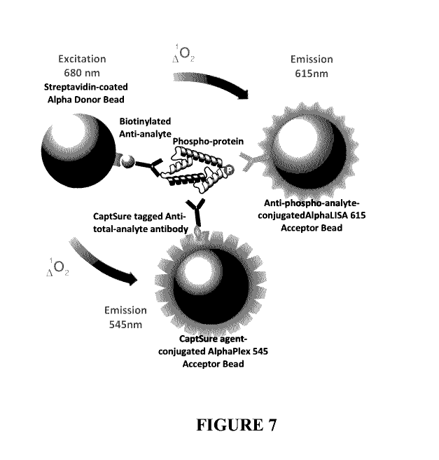

FIG. 7: Single analyte, 2 Epitope target detection system.

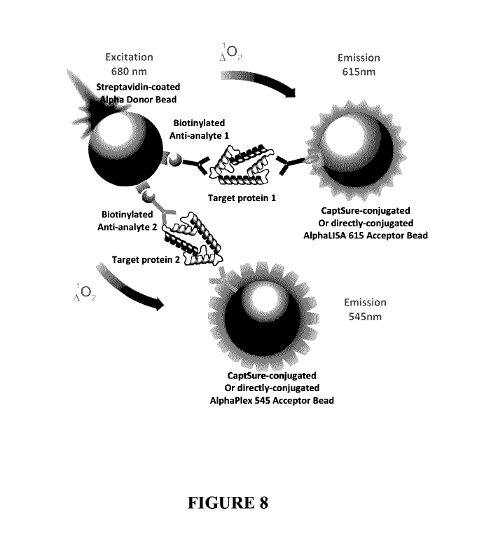

FIG. 8: Dual analyte target detection system.

### DESCRIPTION OF THE EXEMPLARY EMBODIMENTS

Certain embodiments provide an analyte detection system, comprising: i) a first agent bound to a first bead and capable of binding to the analyte; ii) a second agent covalently bound to a plurality of peptide tags and capable of binding to the analyte; and iii) a second bead comprising an anti-peptide agent capable of binding with at least one of the plurality of covalently bound peptide tags.

Certain embodiments provide an analyte detection system, comprising: i) a first agent capable of binding to the analyte; ii) a second agent covalently bound to a plurality of peptide tags and capable of binding to the analyte; iii) a first bead capable of binding with the first agent; and iv) a second bead comprising an anti-peptide agent capable of binding with at least one of the plurality of covalently bound peptide tags.

Certain embodiments provide an analyte detection system, comprising: i) a first agent comprising at least one tag and capable of binding to the analyte; ii) a second agent covalently bound to a plurality of peptide tags and capable of binding to the analyte; iii) a first bead comprising a binder capable of binding with the tagged first agent; and iv) a second bead comprising an anti-peptide agent capable of binding with at least one of the plurality of covalently bound peptide tags.

Certain embodiments provide an analyte detection system, comprising: i) a first agent covalently bound to a plurality of peptide tags and capable of binding to the analyte; ii) a second agent comprising at least one tag and capable of binding to the analyte; iii) a first bead comprising an anti-peptide agent capable of binding with at least one of the plurality of covalently bound peptide tags; and iv) a second bead comprising a binder capable of binding with the tagged first agent.

Certain embodiments provide an analyte detection system, comprising: i) a first agent comprising at least one first tag and capable of binding to the analyte; ii) a second agent comprising at least one second tag and capable of binding to the analyte; iii) a first bead comprising a binder capable of binding with the tagged first agent; and iv) a second bead comprising a binder capable of binding with the tagged second agent.

In certain embodiments, the first agent (or the donor agent of any of the foregoing embodiments) may comprise, for example, an agent that may be capable of binding with the analyte, for example an antibody, inclusive, for example, a monoclonal antibody, a polyclonal antibody, a multivalent antibody, a chimeric antibody, a multispecific antibody, or an antibody fragment thereof; an aptamer; an affimer; a protein; a protein receptor; a protein receptor; a protein ligand; a fusion protein, comprising, for example, an immunoglobulin fusion partner, a fusion partner that stabilizes a receptor or a ligand, or a fusion partner that provides a target for binding; avidin, streptavidin, an avidin derivative, or a streptavidin derivative.

In certain embodiments, the first agent may comprise at least one tag or a plurality of tags. The at least one tag or a plurality of tags that may be attached, conjugated, or covalently bound, to a first agent may be a ligand. In certain embodiments, a plurality of tags comprising in the range of between 1-50 tags may be attached, such as conjugated or covalently bound, to a first agent, for example, a plurality of tags comprising in the range of between 2-50 tags, such as 5-8, 3-7, 2-5, or such as 10-15, 15-20, or 25-50 tags, may be attached, conjugated, or covalently bound, to a first agent. In certain embodiments, no more than 50 tags may be attached, such as conjugated or covalently bound, to a first agent, for example, no more than 12 tags, such as no more than 10, no more than 8, no more than 7, no more than 6, no more than 5, or no more than 3 tags, may be attached, conjugated, or covalently bound, to a first agent. In certain embodiments, one tag may be attached, such as conjugated or covalently bound, to a first agent, for example, 2 tags, such as 3, 4, 5, 8, 10, or 12 tags, may be attached, conjugated, or covalently bound, to a first agent. In certain embodiments, at least one of the plurality of tags of the tagged first agent is a ligand, or the plurality of tags of the tagged first agent are ligand tags, wherein the ligand tag is biotin, a biotin derivative, or a peptide tag.

Suitable ligands that may be attached, conjugated, or covalently bound, to a first agent may include, but are not limited to biotin or derivatives thereof, such as 1-[4-azidosalicylamido]-6-[biotinamido]-hexane; psoralen-PEG3-biotin; or TFPA-PEG3-biotin; a metal chelate, such as copper, nickel, or cobalt; maleic anhydride; maleimide; or a peptide tag, such as a FLAG-tag. In certain embodiments, at least one of the plurality of tags of the tagged first agent is a ligand, or the plurality of tags of the tagged first agent, is or comprises an epitope of the binder attached, conjugated, or covalently bound, to a donor bead, such as a first bead.

In certain embodiments, the ligand attached, conjugated to, or covalently bound to, a first agent may comprise a peptide tag, a polyhistidine tag (His-tag), a HA-tag, a myc-tag, biotin or a derivative thereof including, for example, imino biotin, D-desthiobiotin, DSB-X-biotin, biotin dimers or arylstannylbiotin trimer. Biotin and derivatives thereof may be bound to the first agent by biotinylation. Biotinylation reagents and methods for biotinylation of a target molecule are known in the art and include those described by Hermanson (Bioconjugate Techniques, Academic Press, 2008). Biotinylation may comprise, for example, primary amine biotinylation, sulfhydryl biotinylation, carboxyl biotinylation, or glycoprotein biotinylation. Methods for labeling proteins, RNA, DNA and other molecules with the above ligands are generally known in the art and may include methods described by Wong (Chemistry of Protein Conjugation and Cross-Linking, CRC Press LLC, 1991). In certain embodiments, the ligand attached, conjugated to, or covalently bound to, a first agent, is or comprises a peptide tag.

For example, in certain embodiments, the peptide tag is a FLAG-tag is DYKDDDDK (SEQ ID NO: 1), a peptide tag having the amino acid sequence CDYKDDDDK (SEQ ID NO: 8), a FLAG octapeptide, or is a polypeptide protein tag, that can be attached, conjugated, or covalently bound, to the first antibody, or added to the first antibody, such as attached, conjugated, or covalently bound to the first antibody using Recombinant DNA technology. Suitable peptide tags that may be attached, conjugated, or covalently bound, to a first antibody may include, but are not limited to amino acid sequences having no more than 100 amino acids, for example, no more than 50 amino acids, no more than 30 amino acids, no more than 25 amino acids, such as no more than 20, no more than 15, no more than 12, no more than 10, no more than 9, or no more than 8 amino acids. In certain embodiments, peptide tags that may be attached, conjugated, or covalently bound, to a first antibody may have amino acid sequences in the range of between 5-15 amino acids, for example, in the range of between 6-12 amino acids, such as between 7-10, or between 8-9 amino acids. In certain embodiments, the peptide tag has the amino acid sequence DYKDDDDK (SEQ ID NO: 1) or CDYKDDDDK (SEQ ID NO: 8). In certain embodiments, peptide tags may not be naturally occurring. In certain embodiments, peptide tags may not be naturally occurring in an organism from which a sample is taken.

In certain embodiments, the second agent (or the acceptor agent of any of the foregoing embodiments) may comprise, for example, an agent that may be capable of binding with the analyte, for example an antibody, inclusive, for example, a monoclonal antibody, a polyclonal antibody, a multivalent antibody, a chimeric antibody, a multispecific antibody, or an antibody fragment thereof; an aptamer; an affimer; a protein; a protein receptor; a protein ligand; or a fusion protein, comprising, for example, an immunoglobulin fusion partner, a fusion partner that stabilizes a receptor or a ligand, or a fusion partner that provides a target for binding.

In certain embodiments, the second agent may comprise at least one peptide tag or a plurality of peptide tags covalently bound to the second agent. In certain embodiments, the plurality of peptide tags covalently bound to the second agent may be in the range of between 1-15 peptide tags, such as 2-9, 5-8, 3-7, 2-5, or 10-15, peptide tags covalently bound to the second agent. In certain embodiments, no more than 15 peptide tags may be covalently bound to the second agent, for example, no more than 12 peptide tags, such as no more than 10, no more than 8, no more than 7, no more than 6, no more than 5, or no more than 3 peptide tags, may be covalently bound to the second agent.

Suitable peptide tags that may be covalently bound to a second agent may include, but are not limited to amino acid sequences having no more than 30 amino acids, for example, no more than 25 amino acids, such as no more than 20, no more than 15, no more than 12, no more than 10, no more than 9, or no more than 8 amino acids. In certain embodiments, at least one, a plurality, or each, of the plurality of peptide tags that may be covalently bound to a second antibody may have amino acid sequences in the range of between 5-15 amino acids, for example, in the range of between 6-12 amino acids, such as between 7-10, or between 8-9 amino acids. In certain embodiments, at least one, a plurality, or each, of the plurality of peptide tags covalently bound to the second antibody is or comprises a FLAG-tag having the amino acid sequence DYKDDDDK (SEQ ID NO: 1), a peptide tag having the amino acid sequence CDYKDDDDK (SEQ ID NO: 8), a FLAG octapeptide, or is a polypeptide protein tag. In certain embodiments, for example, the peptide tag has the amino acid sequence DYKDDDDK (SEQ ID NO: 1), or the peptide tag has the amino acid sequence CDYKDDDDK (SEQ ID NO: 8). In certain embodiments, the protein tag, may be covalently bound to the second antibody by using Recombinant DNA technology. In certain embodiments, at least one of the plurality of peptide tags of the tagged second antibody, or the plurality of tags of the tagged second antibody, is or comprises an epitope of the anti-peptide antibody attached, conjugated, or covalently bound, to an acceptor bead, such as a second bead.

Suitable peptide tags that may be covalently bound to a second agent may include, but are not limited to a FLAG-tag, for example DYKDDDDK (SEQ ID NO: 1); a peptide tag having the amino acid sequence KRITVEEALAHPYLEQYYDPTDE (SEQ ID NO: 2); a peptide tag having the amino acid sequence HHHHHH (SEQ ID NO: 3); a peptide tag having the amino acid sequence EQKLISEEDL (SEQ ID NO: 4); a peptide tag having the amino acid sequence YPYDVPDYA (SEQ ID NO: 5); a peptide tag having the amino acid sequence YTDIEMNRLGK (SEQ ID NO: 6); a peptide tag having the amino acid sequence QPELAPEDPED (SEQ ID NO: 7); a peptide tag having the amino acid sequence CDYKDDDDK (SEQ ID NO: 8) a FLAG octapeptide; a polypeptide protein tag; or a polypeptide sequence that does not include a plurality of consecutive amino acids with the same charge. In certain embodiments, at least a portion of the peptide tags may not be naturally occurring. In certain embodiments, the peptide tags do not denature or inactivate the peptide-tagged agent to which they are attached. In certain embodiments, at least one of the peptide tags is more hydrophilic than the FLAG-tag. In certain embodiments, at least a portion of the peptide tags may be removed from the peptide-tagged agent to which they are attached by treatment with the specific proteinase, enterokinase (enteropeptidase). In certain embodiments, at least a portion of the peptide tags may not be naturally occurring in an organism from which a sample is taken. In certain embodiments, peptide tags may include amino acid sequences having no more than 100 amino acids, for example, no more than 50, no more than 30, no more than 20, no more than 15, no more than 12, no more than 10, no more than 9, or no more than 8 amino acids. In certain embodiments, peptide tags may include amino acid sequences having in the range of 4-100 amino acids, for example in the range of 4-50, 4-30, 4-20, 6-15, 6-12, 8-20, 8-15, or in the range of 8-12 amino acids. In certain embodiments, the peptide tags may include one or more of the above features for example, the peptide tags may comprise no more than 15 amino acids, may not be naturally occurring, and/or may be more hydrophilic than the FLAG tag.

In certain embodiments, the peptide tags may also be used in conjunction with other affinity tags, for example a polyhistidine tag (His-tag), HA-tag or myc-tag. In certain further embodiments, for example, the ligand attached, conjugated to, or covalently bound to, a first agent, may comprise one or more of the above tags, for example one or more of the peptide tags may also be used in conjunction with one or more of polyhistidine tag (His-tag), HA-tag or myc-tag.

An agent used in an assay to bind with an analyte may be tagged with a peptide. For example, adding a peptide tag, such as a FLAG-tag, to the first agent allows the first agent to be bound and/or associated with an anti-peptide agent against the peptide tag (for example a peptide tag such as the FLAG-tag sequence). In certain embodiments, a peptide tag, such as a FLAG-tag, may also be used in conjunction with other affinity tags for example a polyhistidine tag (His-tag), HA-tag or myc-tag. The FLAG-tag was the first example of a fully functional epitope tag to be published in the scientific literature (see Hopp et al., Bio/Technology 6: 1204-1210, 1988). Its structure has been optimized for compatibility with the proteins it is attached to, in that it is more hydrophilic than other common epitope tags and therefore less likely to denature or inactivate proteins to which it is appended. In addition, it can be removed readily from proteins by treatment with the specific proteinase, enterokinase (enteropeptidase).

In addition to comprising a ligand attached, conjugated, or covalently bound, to an agent, such as a first agent or a second agent, at one or a plurality of sites, the agent must be capable of binding, capturing, or attaching, to the analyte, such as binding to an epitope of the analyte, for example a phospho-epitope of the analyte. For example, in certain embodiments, the tagged first agent is capable of binding to a first epitope of the analyte and the second agent is capable of binding to a second epitope of the analyte. In certain embodiments, the type of epitope of the first epitope of the analyte and the second epitope of the analyte are the same type of epitope, or are two different types of epitope. In certain embodiments, the first agent and the second agent are the same type of agent. In certain embodiments, the first agent and the second agent are different types of antibodies. In certain embodiments, the first epitope of the analyte and/or the second epitope of the analyte is a phospho-epitope. In certain embodiments, the first epitope of the analyte and the second epitope of the analyte do not overlap but are still in close proximity to each other, or do not overlap and are distal to each other. In certain embodiments, the first epitope of the analyte and the second epitope of the analyte do not overlap and are sufficiently distal to each such that no steric interactions or substantially no steric interactions, are incurred between the first and second agent. In certain embodiments, the epitopes are bound by the first agent and the second agent, respectively. In certain embodiments, the agent may comprise a naturally occurring antibody, or a mutant antibody, or may be protein containing the binding fragment of an antibody or a protein receptor.

In certain embodiments, the analyte detection system, kit, and methods of using the same, employ at least two types of beads, for example, a donor bead and an acceptor bead. For example, in certain embodiments, the analyte detection system, kit, and methods of using the same, employ two or more types of beads comprising a donor first bead and an acceptor second bead. In certain embodiments, approximately the same number of donor and acceptor beads are used. In certain embodiments, the ratio of acceptor bead to donor bead is in the range of between 0.1-10, for example in the range of between 0.1-0.2, between 0.2-0.5, between 0.5-1, between 1-2, between 2-5, or between 5-10. In certain embodiments, the ratio of acceptor bead to donor bead is in the range of between 0.1-10, for example in the range of between 0.2-5, between 0.5-2, between 0.66-1.5, between 0.75-1.33, between 0.8-1.25, between 0.9-1.1, or between 0.95-1.05. In certain embodiments, the analyte detection system, kit, and methods of using the same, employ a plurality of beads, for example, a plurality of donor beads and a plurality acceptor beads, such as a first donor bead, a second donor bead, and a first acceptor bead; a first donor bead, a first acceptor bead, and a second acceptor bead; or a first donor bead, a second donor bead, a first acceptor bead, and a second acceptor bead.

In certain embodiments, the analyte detection system, kit, and methods of using the same, employ at least three types of beads, for example, at least one donor bead and at least a first acceptor bead and a second acceptor bead. In certain embodiments, the ratio of the number of the at least one donor bead present is in the range of 0.1-10, for example in the range of between 0.1-0.2, between 0.2-0.5, between 0.5-1, between 1-2, between 2-5, or between 5-10, relative to the number of the first acceptor bead.

In certain embodiments, the ratio of the number of the at least one donor bead present is in the range of between 0.1-10, for example in the range of between 0.2-5, between 0.5-2, between 0.66-1.5, between 0.75-1.33, between 0.8-1.25, between 0.9-1.1, or between 0.95-1.05, relative to the number of the first acceptor bead. In certain embodiments, the ratio of the number of the at least one donor bead present is in the range of between 1-3, for example in the range of between 1.25-2.75, between 1.5-2.5, between 1.75-2.25, or between 1.95-2.05, relative to the number of the first acceptor bead.

In certain embodiments, the ratio of the number of the at least one donor bead present is in the range of 0.1-10, for example in the range of between 0.1-0.2, between 0.2-0.5, between 0.5-1, between 1-2, between 2-5, or between 5-10, relative to the number of the second acceptor bead. In certain embodiments, the ratio of the number of the at least one donor bead present is in the range of between 0.1-10, for example in the range of between 0.2-5, between 0.5-2, between 0.66-1.5, between 0.75-1.33, between 0.8-1.25, between 0.9-1.1, or between 0.95-1.05, relative to the number of the second acceptor bead. In certain embodiments, the ratio of the number of the at least one donor bead present is in the range of between 1-3, for example in the range of between 1.25-2.75, between 1.5-2.5, between 1.75-2.25, or between 1.95-2.05, relative to the number of the second acceptor bead. In certain embodiments, the ratio of the number of the at least one donor bead present is in the range of 0.1-10, for example in the range of between 0.1-0.2, between 0.2-0.5, between 0.5-1, between 1-2 (for example 2), between 2-5, or between 5-10, relative to the total of the at least a first acceptor bead and a second acceptor bead (for example the total of the number of a first, a second, a third, and a fourth acceptor bead). In certain embodiments, the ratio of the number of the at least one donor bead present is in the range of between 0.1-10, for example in the range of between 0.2-5, between 0.5-2, between 0.66-1.5, between 0.75-1.33, between 0.8-1.25, between 0.9-1.1, or between 0.95-1.05, relative to the total of the at least a first acceptor bead and a second acceptor bead. In certain embodiments, the ratio of the number of the at least one donor bead present is in the range of between 1-3, for example in the range of between 1.25-2.75, between 1.5-2.5, between 1.75-2.25, or between 1.95-2.05, relative to the total of the at least a first acceptor bead and a second acceptor bead.

Suitable beads that may be utilized in the analyte detection system, kit, and methods of using the same, include, but are not limited to a latex bead, a magnetic bead, a bead doped with a photosensitizer capable of emitting singlet oxygen, a bead doped with a photoactive indicator precursor capable of reacting with singlet oxygen to produce a photoactive indicator capable of fluorescing, a bead capable of being stimulated by light, a bead capable of fluorescing, or a bead capable of interacting with a second bead via a chemical transfer interaction, such as via emitting singlet oxygen. See, for example, U.S. Pat. No. 5,807,675, which is incorporated herein by reference in its entirety. In certain embodiments, the beads may have a diameter in the range of between 20 nm and 20 mm, such as between 50-500 nm, between 100-500 nm, between 150-350 nm, 250-350 nm, 250-275 nm, between 280-310 nm, between 290-325 nm, between 310-350 nm, or between 325-350 nm. In certain embodiments, a first type of bead, such as a donor bead, may be capable of interacting with a second type of bead, such as an acceptor bead, via a chemical transfer interaction when the first type of bead and the second type of bead are in close proximity to each other, such as when the beads are disposed within the range of between 15-250 nm, for example, between 15-225 nm, between 25-225 nm, between 50-225 nm, between 100-225 nm, between 150-225 nm, between 100-200 nm, between 25-75 nm, between 50-100 nm, between 175-225 nm, between 190-210 nm, between 225-250 nm, or between 150-200 nm of one another, such as within 25 nm of one another, for example within 50 nm, within 75 nm, within 100 nm, within 125 nm, within 150 nm, within 175 nm, within 180 nm, within 190 nm, within 200 nm, within 210 nm, within 220 nm, within 230 nm, or within 240 nm of one another.

Suitable donor beads that may be utilized in the analyte detection system, kit, and methods of using the same, include, but are not limited to a latex bead, a magnetic bead, a bead doped with a photosensitizer capable of emitting singlet oxygen, a bead capable of being stimulated by light, or a bead capable of interacting via a chemical transfer interaction, such as via emitting singlet oxygen. In certain embodiments, the analyte detection system, kit, and methods of using the same, may utilize two or more types of donor beads, such that each type of donor bead utilized may emit singlet oxygen when stimulated by light at the same wavelength. In certain embodiments, the analyte detection system, kit, and methods of using the same, may utilize two or more types of donor beads, such that light at the different wavelengths stimulates the different types of donor beads to emit singlet oxygen when stimulated, for example, light at a first wavelength stimulates a first type of donor bead to emit singlet oxygen, and light at a second wavelength stimulates a second type of donor bead to emit singlet oxygen. For example, in certain embodiments, the donor bead may emit singlet oxygen when stimulated by light, such as when stimulated by light at a wavelength in the range of between 250-1100 nm, for example, by light at a wavelength in the range of between 300-1000 nm, between 620-700 nm, between 620-650 nm, between 640-700 nm, between 650-700 nm, between 670-690 nm, between 680-700 nm, or between 660-680 nm, such as by light at a wavelength of 620 nm, 630 nm, 640 nm, 650 nm, 660 nm, 670 nm, 675 nm, 680 nm, 685 nm, 690 nm, or 700 nm. For example, in certain embodiments, the analyte detection system, kit, and methods of using the same, may utilize two or more types of donor beads, such that each type of donor bead utilized may emit singlet oxygen when stimulated by light at the same wavelength, such as at a wavelength in the range of between 650-700 nm, for example at 680 nm.

Suitable acceptor beads that may be utilized in the analyte detection system, kit, and methods of using the same, include, but are not limited to a latex bead, a magnetic bead, a bead doped with a photoactive indicator precursor, such as rubrene, europium, a europium chelate, samarium, or terbium, capable of reacting with singlet oxygen to produce a photoactive indicator capable of fluorescing, a bead capable of being stimulated by light, a bead capable of fluorescing, or a bead capable of interacting via a chemical transfer interaction, such as via emitting singlet oxygen. In certain embodiments, the acceptor bead emits light, such as fluoresces, in response to a chemical transfer interaction with a donor bead, such as by an emission of singlet oxygen by the donor bead, when in close proximity to said donor bead. For example, the acceptor bead, such as a bead that is associated with the binding of least one of the plurality of peptide tags covalently bound to the second agent be utilized in the analyte detection system, kit, and methods of using the same, may comprise a photoactive indicator precursor that reacts with singlet oxygen forming a photoactive indicator. In certain embodiments, the analyte detection system, kit, and methods of using the same, may utilize two or more types of acceptor beads, such that when irradiated, the different types of acceptor beads emit a fluorescence at different wavelength, for example, when irradiated a first type of acceptor bead emits a fluorescence at a first wavelength, and when irradiated a second type of acceptor bead emits a fluorescence at a second wavelength. In certain embodiments, the acceptor bead may be irradiated by light at a wavelength in the range of between 250-1100 nm, for example, by light at a wavelength in the range of between 300-1000 nm, between 450-950 nm, between 360-441 nm, between 620-700 nm, between 600-630 nm, between 620-650 nm, between 640-700 nm, between 650-700 nm, between 670-690 nm, between 680-700 nm, or between 660-680 nm, such as by light at a wavelength of 620 nm, 630 nm, 640 nm, 650 nm, 660 nm, 670 nm, 675 nm, 680 nm, 685 nm, 690 nm, or 700 nm.

In certain embodiments, the photoactive indicator on the acceptor bead, when irradiated, may fluoresce (i.e., emit a fluorescence) at a wavelength in the range of between 500-625 nm, such as at a wavelength in the range of between 525-575 nm, between 525-550 nm, between 540-560 nm, between 540-550 nm, 590-620 nm, between 600-625 nm, or 610-620 nm, such as at a wavelength of 520 nm, 530 nm, 535 nm, 540 nm, 545 nm, 550 nm, 555 nm, 560 nm, 600 nm, 605 nm, 610 nm, 615 nm, 620 nm, or 625 nm. For example, when irradiated a first type of acceptor bead may emit a fluorescence at a first wavelength in the range of between 525-575 nm, such as at a first wavelength of 545 nm, and when irradiated a second type of acceptor bead may emit a fluorescence at a second wavelength in the range of between 590-625 nm, such as at a second wavelength of 615 nm.

Suitable binders, coated, attached, conjugated, or bound to a bead, for example, a first bead, such as a donor bead, may include, but are not limited to an antibody, inclusive, for example, a monoclonal antibody, a polyclonal antibody, a multivalent antibody, a chimeric antibody, a multispecific antibody, or an antibody fragment thereof; an aptamer; an affimer; a protein; a protein receptor; a protein receptor; a protein ligand; or a fusion protein, comprising, for example, an immunoglobulin fusion partner, a fusion partner that stabilizes a receptor or a ligand, or a fusion partner that provides a target for binding; avidin, streptavidin, an avidin derivative, or a streptavidin derivative. Derivatives of avidin or streptavidin are known in the art and may include forms of avidin or streptavidin that have been modified to increase their binding affinity to modified and/or unmodified solid substrates or ligands. For example, streptavidin may be modified to add one or more amine groups, histidine residues or sulfhydryl groups to the molecule. In certain embodiments, the derivative of streptavidin may comprise neutravidin, captavidin or streptavidin mutants (e.g. H127C or S139C). In certain embodiments, a hydrophobic or hydrophilic binder may be passively bound to the hydrophobic or hydrophilic solid substrates, respectively. For example, streptavidin (or derivates thereof) may be passively bound to a hydrophobic solid substrate. In certain embodiments, the solid substrate may comprise a linker which facilitates covalent bonding of the binder to the solid substrate. For example, the linker may comprise glutathione, maleic anhydride, a metal chelate, or maleimide. The binder may then be bound to the solid substrate via the linker.

In certain embodiments, the binder is an antibody having an affinity for at least one ligand attached, conjugated, or covalently bound, to an antibody, such as a first antibody. For example, the further antibody binder may have a selective affinity for binding to the at least one, or each of the plurality of ligands attached, conjugated, or covalently bound, to an antibody, such as a first antibody. For example, in certain embodiments, the binder may be a further antibody, such as a second anti-peptide antibody having a selective affinity for at least one peptide tag, or a plurality of peptide tags attached, conjugated, or covalently bound, to a first antibody.

In certain embodiments, the binder is protein A and/or protein G that is coated on the first bead. For example, in certain embodiments, the first bead may be coated with protein A and/or protein G, which are suitable for attaching to an antibody, in a solution. In certain embodiments, however, protein A and/or protein G may provide reduced utility in samples containing endogenous antibodies, such as serum or plasma, as these will block binding of the desired agent(s).

In certain embodiments, the anti-peptide agent coated, attached, conjugated, or bound to a bead, for example, a first bead or a second bead, such as an acceptor bead, may include, but are not limited to an antibody comprising a binding region highly selective for the amino acid sequence, or a portion thereof, of the peptide tag covalently bound to the second agent, such as a peptide tag having the amino acid sequence DYKDDDDK (SEQ ID NO: 1) or CDYKDDDDK (SEQ ID NO: 8). For example, the binding region of the anti-peptide agent may be highly selective for at least 60% of amino acid sequence of the peptide tag covalently bound to the second agent, such as 70%, 80%, 90%, 95%, or 100%, of amino acid sequence of the peptide tag covalently bound to the second agent.

In some embodiments, the first bead or the second bead may be treated with a blocking agent that binds non-specifically to and saturates binding sites to prevent direct binding of unwanted components (e.g. the analyte, non-analyte antibodies present in a sample) to the excess sites on the first or second bead. Examples of blocking agents may include, for example, gelatin, BSA, egg albumin, casein, and non-fat milk. In some embodiments, the solid substrate and/or the bound immobilisation agent may be treated with the blocking agent prior to the addition of the capture agent or concurrent with the addition of the capture agent.

Certain embodiments provide a method for detecting an analyte, comprising: i) binding an analyte with a first agent and a second agent, wherein: a) the first agent comprises one or more first tags; and b) the second agent comprises a plurality of covalently bound peptide second tags; ii) attaching at least one of the one or more first tags to a binder conjugated to a first bead; iii) binding at least one of the plurality of covalently bound peptide second tags to an anti-peptide agent conjugated to a second bead; and iv) detecting the presence of the analyte by sensing an emission resulting from an interaction between the attached first bead and the bound second bead.

In certain embodiments, the binding an analyte with a first agent and a second agent forms an analyte complex, and the resulting analyte complex is then immobilized to two different types of beads, comprising a first bead, such as an acceptor bead, and a second bead, such as a donor bead. In certain embodiments, the first bead is an acceptor bead and the second bead is a donor bead. For example, in certain embodiments, the method may further comprise attaching at least one of the one or more first tags to a binder conjugated to a first bead, and binding at least one of the plurality of covalently bound peptide second tags to an antibody conjugated to a second bead. The resulting analyte complex, now attached to a first bead and bound to a second bead, is available for detecting the presence of the analyte by sensing an emission resulting from an interaction between the attached first bead and the bound second bead. For example, the first bead, now in close proximity to the second bead via the attached and bound analyte complex, and having a photosensitizer contained within the bead may emit singlet oxygen when irradiated with light. The singlet oxygen produced from the first bead may react with a photoactive indicator precursor contained within the second bead because of its close proximity to produce a photoactive indicator that is capable of fluorescing when irradiated with light.

In certain embodiments, the detection method may further include exciting a photosensitizer contained within or coated on a donor bead by irradiation with light to produce singlet oxygen that reacts with a photoactive indicator precursor contained within or coated on an acceptor bead to produce said photoactive indicator within or on the acceptor bead. In certain embodiments, the detection method may further include irradiating the produced photoactive indicator within or on the acceptor bead and measuring the fluorescence emitted by said photoactive indicator.

Certain embodiments include at least one, such as a plurality, of detection complexes dispersed in the sample and method of and kits for making the same, wherein the at least one detection complex comprises (1) an analyte complex, such as a sandwich complex, comprising the analyte bound to a first agent comprising one or more first tags and bound separately, for example, directly bound to a second agent covalently bound to a plurality of peptide; (2) a first bead having a plurality of binders conjugated thereon, wherein at least one of the one or more first tags present from the one or more analyte complexes is bound to one of the plurality of binders; and (3) a second bead having a plurality of anti-peptide agents (for example a plurality of anti-peptide antibodies) conjugated thereon, wherein at least one of the plurality of covalently bound peptide second tags from the one or more analyte complexes is bound to one of the plurality of anti-peptide agents; wherein the first beads are capable of a chemical transfer interactions with the second beads. In certain embodiments, the at least one detection complex further comprises at least a second analyte complex, for example 2-5, 2-3, 2-4, or 3-5 analyte complexes. For example, there could be 2-5 analyte complexes bound between a first bead and a second bead. Certain embodiments comprise irradiating the donor beads in at least a portion of the detection complexes with light at an appropriate wavelength to trigger excitation of at least a portion of the acceptor beads in the detection complexes and, upon irradiation of the at least a portion of the acceptor beads, fluorescence of the at least a portion of the acceptor beads. In certain embodiments, the method further comprises detecting light emitted by the acceptor beads.

Certain embodiments provide a method of detecting the presence of, or determining the absence of, a specific analyte in a sample, may include detecting analyte with two beads in the presence of a plurality of non-analyte antibodies in a sample, may include detecting at least two different analytes in a sample, or may include detecting at least two epitopes of an analyte in a sample. For example, in certain embodiments, the absence of a specific analyte is determined when no fluorescence within a detection range is sensed upon irradiation of the first bead and the second bead.

In certain embodiments a method may include providing the analyte in a sample that contains multiple components, such as two different types of analytes, or a plurality of analyte types in a sample, such as biological sample. For example, in certain embodiments, the specific analyte to be detected may be contained within a sample that also contains non-analyte antibodies. In certain embodiments, the binder preferentially binds to the first agent over the non-analyte antibodies, and the anti-peptide agent preferentially binds to the second agent covalently bound to a plurality of peptide tags. In certain embodiments, the first agent may bind to one epitope on an analyte and the second agent covalently bound to a plurality of peptide tags may bind to a second epitope on the same analyte.

In certain embodiments the methods may include mixing the sample, the tagged first agent, the tagged second agent, and at least one or a plurality of second beads, followed by adding at least one or a plurality of first beads, such as a donor bead. In certain embodiments the methods may include incubating the sample, the tagged first agent, the tagged second agent, and at least one or a plurality of second beads for approximately 1 hour, followed by adding at least one or a plurality of first beads, such as a donor bead, and further incubating for approximately 1 hour. In certain embodiments, the addition of the at least one or a plurality of first beads and the at least one or a plurality of second beads to the mixture may be simultaneously, or sequentially. In certain embodiments, the second bead is added to the mixture, prior to, concurrently with, or after the second bead, and the resulting mixture is incubated for a period of time, for example, for less than 30 minutes, for less than one hour, for approximately one hour, for more than one hour, for less than two hours. In certain embodiments, the method comprises mixing the second bead with the incubated mixture, in the presence or absence of the first bead, and incubating the resulting mixture for a period of time, for example, for less than 30 minutes, for less than one hour, for approximately one hour, for more than one hour, for less than two hours. In certain embodiments, the first bead is added to the mixture, prior to, concurrently with, or after the second bead, and the resulting mixture is incubated for a period of time, for example, for less than 30 minutes, for less than one hour, for approximately one hour, for more than one hour, for less than two hours. In certain embodiments, the method comprises mixing the first bead with the incubated mixture, in the presence or absence of the second bead, and incubating the resulting mixture for a period of time, for example, for less than 30 minutes, for less than one hour, for approximately one hour, for more than one hour, for less than two hours. In certain embodiments, the sample, the tagged first agent, the tagged second agent, and the second bead, in the presence or the absence of the first bead, are incubated for a period of time, for example, for approximately one hour, for less than one hour, for greater than one hour, for less than two hours, or for approximately two hours.

In certain embodiments, the concentration of the binder attached, conjugated, or covalently bound to the first bead, is at least as large as the concentration of the tagged first agent in the mixture. In certain embodiments, the concentration of the binder attached, conjugated, or covalently bound to the first bead, is at least 2 times greater than the concentration of the tagged first agent in the mixture, for example, the concentration of the binder is at least 3 times greater, such as at least 4 times greater, at least 5 times greater, at least 7 times greater, at least 9 times greater, or at least 10 times greater than the concentration of the tagged first agent in the mixture. In certain embodiments, the concentration of the binder attached, conjugated, or covalently bound to the first bead, is in the range of between 2-15 times greater than the concentration of the tagged first agent in the mixture, for example, the concentration of the binder is in the range of between 2-10 times greater, such as between 2-8 times greater, between 2-5 times greater, between 4-12 times greater, between 3-9 times greater, between 4-8 times greater, or between 10-15 times greater, than the concentration of the tagged first agent in the mixture.

In certain embodiments, the binder attached, conjugated, or covalently bound to the first bead may be present at a ratio of at least 100:1, 125:1, 150:1, 200:1, 250:1, 300:1, 400:1, 500:1, 750:1, or 1000:1, on a weight:weight basis, relative to the tagged first agent in the mixture. In certain embodiments, the binder may be present at a ratio of in a range of 100:1-1000:1, 100:1-500:1, 100:1-300:1, 250:1-300:1, 300:1-1000:1, 300:1-500:1, 300:1-400:1, 300:1-350:1, 325:1-375:1, 300:1-310:1, 310:1-320:1, 320:1-330:1, 330:1-340:1, 340:1-350:1, or 350:1-360:1, on a weight:weight basis, relative to the tagged first agent in the mixture.

In certain embodiments, the binder attached, conjugated, or covalently bound to the first bead may be present at a ratio of at least 100:1, 125:1, 150:1, 200:1, 250:1, 300:1, 400:1, 500:1, 750:1, or 1000:1, on a mole:mole basis, relative to the tagged first agent in the mixture. In certain embodiments, the binder may be present at a ratio of in a range of 100:1-1000:1, 100:1-500:1, 100:1-300:1, 250:1-300:1, 300:1-1000:1, 300:1-500:1, 300:1-400:1, 300:1-350:1, 325:1-375:1, 300:1-310:1, 310:1-320:1, 320:1-330:1, 330:1-340:1, 340:1-350:1, or 350:1-360:1, on a mole:mole basis, relative to the tagged first agent in the mixture.

In certain embodiments, the concentration of the anti-peptide agent, such as the anti-peptide agent attached, conjugated, or covalently bound to the second bead, is at least as large as the concentration of the tagged first agent in the mixture. In certain embodiments, the concentration of the anti-peptide agent, such as the anti-peptide agent attached, conjugated, or covalently bound to the second bead, is at least 2 times greater than the concentration of the tagged second agent in the mixture, for example, the concentration of the anti-peptide agent is at least 3 times greater, such as at least 4 times greater, at least 5 times greater, at least 7 times greater, at least 9 times greater, or at least 10 times greater than the concentration of the tagged second agent in the mixture. In certain embodiments, the concentration of the anti-peptide agent, such as the anti-peptide agent attached, conjugated, or covalently bound to the second bead, is in the range of between 2-15 times greater than the concentration of the tagged second agent in the mixture, for example, the concentration of the anti-peptide agent is in the range of between 2-10 times greater, such as between 2-8 times greater, between 2-5 times greater, between 4-12 times greater, between 3-9 times greater, between 4-8 times greater, or between 10-15 times greater, than the concentration of the tagged second agent in the mixture.

In certain embodiments, the anti-peptide agent may be present at a ratio of at least 100:1, 125:1, 150:1, 200:1, 250:1, 300:1, 400:1, 500:1, 750:1, or 1000:1, on a weight:weight basis, relative to the tagged second agent in the mixture. In certain embodiments, the binder may be present at a ratio of in a range of 100:1-1000:1, 100:1-500:1, 100:1-300:1, 250:1-300:1, 300:1-1000:1, 300:1-500:1, 300:1-400:1, 300:1-350:1, 325:1-375:1, 300:1-310:1, 310:1-320:1, 320:1-330:1, 330:1-340:1, 340:1-350:1, or 350:1-360:1, on a weight:weight basis, relative to the tagged second agent in the mixture.

In certain embodiments, the anti-peptide agent may be present at a ratio of at least 100:1, 125:1, 150:1, 200:1, 250:1, 300:1, 400:1, 500:1, 750:1, or 1000:1, on a mole:mole basis, relative to the tagged second agent in the mixture. In certain embodiments, the binder may be present at a ratio of in a range of 100:1-1000:1, 100:1-500:1, 100:1-300:1, 250:1-300:1, 300:1-1000:1, 300:1-500:1, 300:1-400:1, 300:1-350:1, 325:1-375:1, 300:1-310:1, 310:1-320:1, 320:1-330:1, 330:1-340:1, 340:1-350:1, or 350:1-360:1, on a mole:mole basis, relative to the tagged second agent in the mixture.

In certain embodiments, the concentration of the anti-peptide agent, such as the anti-peptide agent attached, conjugated, or covalently bound to a first bead or a second bead, is less than 5000 nanograms of the anti-peptide agent per milliliter of solution, for example, less than 2500 ng/ml, less than 2000 ng/ml, less than 1500 ng/ml, less than 1000 ng/ml, less than 500 ng/ml, less than 400 ng/ml, less than 300 ng/ml, less than 250 ng/ml, less than 200 ng/ml, less than 100 ng/ml, or less than 50 ng/ml. In certain embodiments, the concentration of the anti-peptide agent, such as the anti-peptide agent attached, conjugated, or covalently bound to the second bead, is in the range of between 1-500 ng/ml, for example, between 1-400 ng/ml, between 1-300 ng/ml, between 1-250 ng/ml, between 1-200 ng/ml, between 1-100 ng/ml, or between 1-50 ng/ml. In certain embodiments, the concentration of the anti-peptide agent, such as the anti-peptide agent attached, conjugated, or covalently bound to a first bead or a second bead, is a concentration of up to 10 μg/ml, for example, up to 1 μg/ml, 5 μg/ml, or 8 μg/ml.

In certain embodiments, the concentration of the analyte is in the range of between 1-100 ng/ml, for example, between 1-50 ng/ml, between 1-25 ng/ml, between 10-50 ng/ml, between 1-10 ng/ml. In certain embodiments, the concentration of the analyte is below 1 ng/mL. In certain embodiments, the concentration of the analyte is above 50 ng/mL. In certain embodiments, the concentration of the analyte is 100 ng/ml or less, 10 ng/ml or less, 1 ng/ml or less, 100 pg/ml or less, or 10 pg/ml or less, 1 pg/ml or less, 100 fg/ml or less, 10 fg/ml or less, or 1 fg/ml or less. In certain embodiments, the concentration of the analyte is greater than 10 ng/ml, greater than 1 ng/ml, greater than 100 pg/ml or, greater than 10 pg/ml, greater than 1 pg/ml, greater than 100 fg/ml, greater than 10 fg/ml or greater than 1 fg/ml. In certain embodiments, the concentration of the analyte is between 1 fg/ml to 100 ng/ml, 1 fg/ml to 10 ng/ml, 1 fg/ml to 1 ng/ml, 10 fg/ml to 100 ng/ml, 10 fg/ml to 10 ng/ml, 10 fg/ml to 1 ng/ml, 100 fg/ml to 100 ng/ml, 100 fg/ml to 10 ng/ml, 100 fg/ml to 1 ng/ml, 1 pg/ml to 100 ng/ml, 1 pg/ml to 10 ng/ml, or 1 pg/ml to 1 ng/ml.

In certain embodiments, the affinity, or rate constant, for binding of the binder to the first agent is approximately the same as the affinity, or rate constant, for binding of the binder to any of the non-analyte antibodies. In certain embodiments, the affinity, or rate constant, for binding of the binder to the first agent is at least 2 times larger than the affinity, or rate constant, for binding of the binder to any of the non-analyte antibodies. For example, the affinity, or rate constant, for binding of the binder to the first agent is at least 5 times larger, such as at least 10 times larger, at least 25 times larger, at least 50 times larger, at least 75 times larger, or at least 100 times larger than the affinity, or rate constant, for binding of the binder to any of the non-analyte antibodies.

In certain embodiments, the affinity, or rate constant, for binding of the anti-peptide agent to the tagged second agent is approximately the same as the affinity, or rate constant, for binding of the binder to any of the non-analyte antibodies. In certain embodiments, the affinity, or rate constant, for binding of the anti-peptide agent to the tagged second agent is at least 2 times larger than the affinity, or rate constant, for binding of the binder to any of the non-analyte antibodies. For example, the affinity, or rate constant, for binding of the anti-peptide agent to the tagged second agent is at least 5 times larger, such as at least 10 times larger, at least 25 times larger, at least 50 times larger, at least 75 times larger, or at least 100 times larger than the affinity, or rate constant, for binding of the binder to any of the non-analyte antibodies.

In certain embodiments, the dissociation constant for binding of the anti-peptide agent to the tagged second agent is approximately the same as the dissociation constant for binding of the binder to any of the non-analyte antibodies. In certain embodiments, the dissociation constant for binding of the anti-peptide agent to the tagged second agent is at least 2 times smaller than the dissociation constant for binding of the binder to any of the non-analyte antibodies. For example, the dissociation constant for binding of the anti-peptide agent to the tagged second agent is at least 5 times smaller, such as at least 10 times smaller, at least 25 times smaller, at least 50 times smaller, at least 75 times smaller, or at least 100 times smaller than the dissociation constant for binding of the binder to any of the non-analyte antibodies.

In certain embodiments, the plurality of tagged first agents and/or the plurality of second agents are stored at a predetermined temperature, for example, at 4° C., room temperature, less than 10° C., less than 20° C., or less than 30° C.

In certain embodiments, the tagged first agents may be stored in or introduced into a first liquid buffer and the tagged second agents may be stored in or introduced into a second liquid buffer.

In certain embodiments, the beads, such as the first bead or the second bead, may be stored under low light conditions, such as in a sealed container. In certain embodiments, the donor beads may be stored under low light conditions, such as in a sealed container.

### EXAMPLES

### Example 1—Comparison of Analyte Detection Using Peptide/Anti-Peptide and Direct Conjugation Systems

A series of pERK analyte detection experiments were conducted employing a biotinylated anti-pERK IgG under comparable conditions. In Example 1 (the peptide/anti-peptide experiment), anti-total ERK IgG was tagged with a plurality of amino acid peptide tags, specifically CDYKDDDDK (SEQ ID NO: 8), and the acceptor beads were conjugated with mouse monoclonal IgG1 anti-peptide agent. In Comparative Examples A and B, the anti-total ERK IgG was directly conjugated onto the acceptor beads. The concentrations of the anti-total ERK IgG were as noted in Table 1 while all other variables were held constant.

TABLE 1

| | Concentration of Anti-Total ERK IgG |
|---|---|
| Example | (ng/ml of sample) |
| Example 1 | 250 |
| Comparative Example A | 2500 |
| Comparative Example B | 500 |

Following incubation, the biotinylated anti-pERK IgG was bound to streptavidin-coated photosensitizer donor beads while, in Example 1, the tagged anti-total ERK IgG was additionally bound via the mouse monoclonal IgG1 anti-peptide agent to the photoactive precursor acceptor beads. The amount of signal detected for each example is presented in FIG. 1.

### Examples 2-3—Effect of Non-Analyte Antibodies

A series of experiments were conducted on samples of protein lysates of serum-activated HEK-293 cells in the absence of any non-analyte antibodies and again in the presence of non-analyte rabbit antibodies at a fixed concentration of 10 mg/ml and analyzed for p-AKT 1/2/3 (Ser473) analyte under comparable conditions and concentrations. Example 2 (no non-analyte antibody) and 3 (spiked with non-analyte antibody) were conducted employing a peptide tagged antibody with complementary antibody conjugated on the acceptor beads and Comparative Examples C (no non-analyte antibody) and D (spiked with non-analyte antibody) repeated the same assays but employed an untagged antibody and protein A-coated acceptor beads. The signal to background signal ratios recorded are presented in FIG. 2.

### Examples 4-11—Detection of Multiple Analytes

Phosphoprotein and total protein analytes were assayed in triplicate from a single lysate sample by delivering portions of the sample to individual culture wells of an assay plate and using biotinylated antibodies and peptide-tagged antibodies to bind the analyte. Table 2 lists the protein detected in each of Examples 4-11.

TABLE 2

| Example | Target |
|---|---|
| Example 4 | AKT (pS473) |
| Example 5 | AKT (pT308) |
| Example 6 | ERK (pT202 Y204) |
| Example 7 | P70S6K (pT389) |
| Example 8 | eIF4E (pS209) |
| Example 9 | CREB (pS133) |
| Example 10 | Total AKT1 |
| Example 11 | Total ERK |

During incubation, the peptide-tagged antibodies, tagged with a plurality of tags each having the amino acid sequence CDYKDDDDK (SEQ ID NO: 8), were bound with mouse monoclonal IgG1 anti-peptide antibodies conjugated to photoactive precursor acceptor beads while the biotinylated antibodies were bound to streptavidin-coated photosensitizer donor beads.

To produce the cells for the lysate sample, individual MCF-7 cells were plated overnight at 200K cells/ml in 200 ml MEM+10% FCS. Cells were then serum starved using MEM+0% FCS for 2 hours, and stimulated for 30 minutes with a dose range of insulin. The amount of signal detected in each of Examples 4-11 is presented in FIG. 3.

### Example 12—Multiplex Detection of Multiple Epitopes

A cell lysate was prepared as follows: MCF-7 cells were plated overnight at 200K/ml in 200 mL MEM+10% FCS. Cells were then treated for 2 hours with varying concentrations of wortmannin in MEM+1% FBS, and then stimulated for 30 min with a dose range of insulin, and then lysed.

The cell lysate was incubated in a multi-well plate with the components listed in Table 3 to measure AKT (pS473) and total AKT1 protein targets.

TABLE 3
Assay Components

Anti-AKT (pS473) antibodies bound to Europium acceptor
beads
Peptide-tagged anti-total AKT1 antibodies
Mouse monoclonal IgG1 anti-peptide antibodies bound to
Terbium acceptor beads
Biotinylated anti-total AKT1 antibodies
Streptavidin-coated photosensitizer donor beads

Detection signals for AKT (pS473) and total AKT1 in the incubated cell lysate were measured using a plate reader at the indicated wortmannin concentrations as shown in FIG. 4.

### Example 13—Multiplex Detection of Multiple Analytes

A cell lysate was prepared as follows: MCF7 cells were plated overnight at 40K cells/well in a 96 well plate in 10% fetal bovine serum at 37° C. in 95% air/5% CO₂. Cells were then treated with wortmannin for 2 hours at the concentrations shown in FIG. 5, and stimulated for 10 minutes with a dose range of insulin, and then lysed.

The cell lysate was incubated in a multi-well plate with the components listed in Table 4 to measure lysate AKT (pS473) and ERK (pT202 Y204) protein targets.

TABLE 4
Assay Components

Anti-AKT (pS473) antibodies directly conjugated to
Europium acceptor beads
Peptide-tagged anti-ERK (pT202 Y204) antibodies
Mouse monoclonal IgG1 anti-peptide antibodies bound to
Terbium acceptor beads
Biotinylated anti-total ERK antibodies
Streptavidin-coated photosensitizer donor beads
Biotinylated anti-total AKT1 antibodies

Detection signals for AKT (pS473) and ERK (pT202 Y204) in the incubated cell lysate were measured using a plate reader as shown in FIG. 5.

### Example 14—Multiplex Detection of Multiple Analytes

A cell lysate was prepared as follows: MCF7 cells were plated overnight at 40K cells/well in a 96 well plate in 10% fetal bovine serum at 37° C. in 95% air/5% CO₂. Cells were then serum starved for 2 hours and treated with insulin for 10 minutes at the concentrations shown in FIG. 6, and then lysed.

The cell lysate was incubated in a multi-well plate with the components listed above in Table 4 to measure lysate AKT (pS473) and ERK (pT202 Y204) protein targets.

Detection signals for AKT (pS473) and ERK (pT202 Y204) in the incubated cell lysate were measured using a plate reader as shown in FIG. 6.

### Prophetic Examples:

As illustrated in FIG. 7, a single target analyte detection system comprising an AlphaLISA 615 Acceptor Bead™ directly-conjugated with an antibody to a phosphorylated site on the target protein, wherein the AlphaPlex 545 Acceptor Bead™ is coated with the CaptSure Agent™, which binds the CaptSure tagged anti-total antibody, and wherein the Alpha Donor bead binds the biotinylated anti-total antibody.

As illustrated in FIG. 8, a dual target analyte detection system comprising an AlphaLISA 615 Acceptor Bead™ and the AlphaPlex 545 Acceptor Bead™ each binding distinct proteins via specific antibodies associated with the bead, wherein one of the Acceptor beads will be directly conjugated to the antibody, and the other will bind the antibody via the CaptSure Agent™, and wherein the type of linkage will depend on the specific AlphaPlex SureFire Ultra kit being used.

All publications and patent applications mentioned in this specification are herein incorporated by reference to the same extent as if each individual publication or patent application was specifically and individually indicated to be incorporated by reference.

While preferred embodiments have been shown and described herein, it will be obvious to those skilled in the art that such embodiments are provided by way of example only. It is intended that the following claims define the scope of the invention and that methods and structures within the scope of these claims and their equivalents be covered thereby.

### Exemplary Embodiments

In an embodiment, an analyte detection system comprises:
i) a first agent comprising at least one tag and capable of binding to the analyte;
ii) a second agent covalently bound to a plurality of peptide tags and capable of binding to the analyte;
iii) a first bead comprising a binder capable of binding with the tagged first agent; and
iv) a second bead comprising an anti-peptide agent capable of binding with at least one of the plurality of covalently bound peptide tags.

In an embodiment, an analyte detection system comprises:
i) a plurality of first antibodies capable of binding to the analyte, wherein each of the first antibodies comprises:
   a) a first region capable of binding with a first antigenic aspect of the analyte; and
   b) a plurality of first tags;
ii) a plurality of second antibodies capable of separately binding to the analyte, wherein each of the plurality of second antibodies comprises:
   a) a second region capable of binding with a second antigenic aspect of the analyte; and
   b) a plurality of covalently bound peptide second tags;
iii) a first bead comprising a plurality of binders conjugated thereon, wherein at least one of the plurality of binders is capable of selectively binding with at least one of the plurality of first tags; and
iv) a second bead comprising a plurality of anti-peptide antibodies conjugated thereon, wherein:
   a) at least one of the anti-peptide antibodies is capable of selectively binding with a least one of the plurality of covalently bound peptide second tags; and
   b) the second bead is capable of detectably interacting with the first bead when the first bead and the second bead are disposed within a certain distance of one another.

In an embodiment, an analyte detection system comprises:
i) a plurality of first agents capable of binding to the analyte, wherein each of the plurality of first agents comprises:
   a) a first region capable of binding with a first antigenic aspect of the analyte; and
   b) a plurality of first tags;
ii) a plurality of second agents capable of separately binding to the analyte, wherein each of the plurality of second agents comprises:
   a) a second region capable of binding with a second antigenic aspect of the analyte; and
   b) a plurality of covalently bound peptide second tags;
iii) a first bead comprising a plurality of binders conjugated thereon, wherein at least one of the plurality of binders is capable of selectively binding with at least one of the plurality of first tags; and
iv) a second bead comprising a plurality of anti-peptide agents conjugated thereon, wherein:
   a) at least one of the anti-peptide agents is capable of selectively binding with a least one of the plurality of covalently bound peptide second tags; and
   b) the second bead is capable of detectably interacting with the first bead when the first bead and the second bead are disposed within a certain distance of one another.

In an embodiment, a system to detect at least two analytes comprises:
i) a donor antibody capable of binding to a first analyte;
ii) a binder conjugated to a donor bead, said binder capable of binding to the donor antibody;
iii) a tagged further donor antibody capable of binding to a second analyte;
iv) a further binder conjugated to the donor bead, said further binder capable of binding to the tagged further donor antibody;
v) a tagged acceptor antibody capable of binding to the analyte, the tagged acceptor antibody comprising a plurality of covalently bound peptide tags; and
vi) a first acceptor bead conjugated to an anti-first peptide antibody, the anti-first peptide antibody capable of binding with at least one of the plurality of covalently bound peptide tags;
vii) a further acceptor antibody capable of binding with the second analyte; and
viii) a second acceptor bead capable of binding with the further acceptor antibody.

In an embodiment, a system to detect at least two analytes comprises:
i) a donor agent capable of binding to a first analyte;
ii) a binder conjugated to a donor bead, said binder capable of binding to the donor agent;
iii) a tagged further donor agent capable of binding to a second analyte;
iv) a further binder conjugated to the donor bead, said further binder capable of binding to the tagged further donor agent;
v) a tagged acceptor agent capable of binding to the analyte, the tagged acceptor agent comprising a plurality of covalently bound peptide tags; and
vi) a first acceptor bead conjugated to an anti-first peptide agent, the anti-first peptide agent capable of binding with at least one of the plurality of covalently bound peptide tags;
vii) a further acceptor agent capable of binding with the second analyte; and
viii) a second acceptor bead capable of binding with the further acceptor agent.

In an embodiment, an analyte detection kit comprises:
i) a first agent comprising at least one tag and capable of binding to the analyte;
ii) a second agent covalently bound to a plurality of peptide tags and capable of binding to the analyte;
iii) a first bead comprising a binder capable of binding with the tagged first agent; and
iv) a second bead comprising an anti-peptide agent capable of binding with at least one of the plurality of covalently bound peptide tags.

In an embodiment, an analyte detection kit comprises:
i) a first agent comprising at least one tag and capable of binding to the analyte;
ii) a second agent covalently bound to a plurality of peptide tags and capable of binding to the analyte;
iii) a donor bead comprising a binder capable of binding with the tagged first agent; and
iv) an acceptor bead comprising an anti-peptide agent capable of binding with at least one of the plurality of covalently bound peptide tags.

In an embodiment, a series of two complementary analyte detection kits that may be used to detect a first and a second analyte within the same sample comprises:

A) a first kit, comprising:
i) a first agent comprising at least one tag and capable of binding to the first analyte;
ii) a second agent covalently bound to a plurality of peptide tags and capable of binding to the first analyte;
iii) a donor bead comprising a binder capable of binding with the tagged first agent of the first kit; and
iv) an acceptor bead comprising an anti-peptide agent capable of binding with at least one of the plurality of covalently bound peptide tags present on the second agent of the first kit, and

B) a second kit, comprising:
i) a first agent comprising at least one tag and capable of binding to the second analyte;
ii) a second agent covalently bound to a plurality of peptide tags and capable of binding to the second analyte;
iii) a donor bead comprising a binder capable of binding with the tagged first agent of the second kit; and
iv) an acceptor bead comprising an anti-peptide agent capable of binding with at least one of the plurality of covalently bound peptide tags present on the second agent of the second kit.

In an embodiment, a series of two complementary analyte detection kits that may be used to detect a first and a second analyte within the same sample comprises:

A) a first kit, comprising:
i) a first agent comprising at least one tag and capable of binding to the first analyte;
ii) a second agent covalently bound to a plurality of peptide tags and capable of binding to the first analyte;
iii) a donor bead comprising a binder capable of binding with the tagged first agent of the first kit; and
iv) an acceptor bead comprising an anti-peptide agent capable of binding with at least one of the plurality of covalently bound peptide tags present on the second agent of the first kit, and

B) a second kit, comprising:
i) a first agent comprising at least one tag and capable of binding to the second analyte;
ii) a second agent covalently bound to a plurality of peptide tags and capable of binding to the second analyte;
iii) a donor bead comprising a binder capable of binding with the tagged first agent of the second kit; and
iv) an acceptor bead comprising an anti-peptide agent capable of binding with at least one of the plurality of covalently bound peptide tags present on the second agent of the second kit, wherein the analyte detection signal from the first kit is different from the analyte detection signal from the second kit.

In an embodiment, a multi-analyte detection kit comprises:
i) a first agent comprising at least one tag and capable of binding to a first analyte;
ii) a second agent covalently bound to a plurality of peptide tags and capable of binding to the first analyte;
iii) a donor bead comprising a binder capable of binding with the tagged first agent; and
iv) an acceptor bead comprising an anti-peptide agent capable of binding with at least one of the plurality of covalently bound peptide tags present on the second agent;
v) a third agent comprising at least one tag and capable of binding to a second analyte;
vi) a fourth agent covalently bound to a plurality of peptide tags and capable of binding to the second analyte;
vii) a further donor bead comprising a binder capable of binding with the tagged third agent; and
viii) a further acceptor bead comprising an anti-peptide agent capable of binding with at least one of the plurality of covalently bound peptide tags present on the fourth agent.

In an embodiment, a multi-analyte detection kit comprises:
i) a first agent comprising at least one tag and capable of binding to a first analyte;
ii) a second agent covalently bound to a plurality of peptide tags and capable of binding to the first analyte;
iii) a donor bead comprising a binder capable of binding with the tagged first agent; and
iv) an acceptor bead comprising an anti-peptide agent capable of binding with at least one of the plurality of covalently bound peptide tags present on the second agent;
v) a third agent comprising at least one tag and capable of binding to a second analyte;
vi) a fourth agent covalently bound to a plurality of peptide tags and capable of binding to the second analyte;
vii) a further donor bead comprising a binder capable of binding with the tagged third agent; and
viii) a further acceptor bead comprising an anti-peptide agent capable of binding with at least one of the plurality of covalently bound peptide tags present on the fourth agent, wherein the acceptor bead and the further acceptor bead emit different signals.

In an embodiment, an analyte detection kit comprises:
i) a first bead comprising a binder capable of binding with a tagged first agent;
ii) a plurality of peptide tags capable of being covalently bound to a second agent, wherein the second agent is capable of binding to a specific analyte; and
iii) a second bead comprising an anti-peptide agent capable of binding with at least one of the plurality of peptide tags capable of being covalently bound to a second agent.

In an embodiment, a method for detecting an analyte comprises:
i) binding an analyte with a first agent and a second agent, wherein:
   a) the first agent comprises one or more first tags; and
   b) the second agent comprises a plurality of covalently bound peptide second tags;
ii) attaching at least one of the one or more first tags to a binder conjugated to a first bead;
iii) binding at least one of the plurality of covalently bound peptide second tags to an anti-peptide agent conjugated to a second bead; and
iv) detecting the presence of the analyte by sensing an emission resulting from an interaction between the attached first bead and the bound second bead.

In an embodiment, a method for detecting an analyte comprises:
i) binding a tagged first agent to the analyte and separately binding a tagged second agent to the analyte, wherein:
   a) the tagged first agent comprises one or more first tags; and
   b) the tagged second agent comprises a plurality of covalently bound peptide second tags;
ii) attaching at least one of the one or more first tags to a binder conjugated to a first bead;
iii) binding at least one of the plurality of covalently bound peptide second tags to an anti-peptide agent conjugated to a second bead; and
iv) detecting the presence of the analyte by sensing an emission resulting from an interaction between the attached first bead and the bound second bead.

In an embodiment, a method for detecting an analyte comprises:
i) binding a first agent to the analyte and separately binding a tagged second agent to the analyte, wherein the tagged second agent comprises a plurality of covalently bound peptide tags;
ii) attaching the first agent to a binder conjugated to a first bead;
iii) binding at least one of the plurality of covalently bound peptide tags to an anti-peptide agent conjugated to a second bead; and
iv) detecting the presence of the analyte by sensing an emission from the bound second bead resulting from an interaction between the attached first bead and said bound second bead.

In an embodiment, a method for detecting an analyte comprises:
i) forming an analyte complex, wherein the analyte complex comprises:
   a) a first agent comprising one or more first tags and bound to the analyte; and
   b) a second agent covalently bound to a plurality of peptide tags and bound to said analyte;
ii) attaching at least one of the one or more first tags to a binder conjugated to a first bead;
iii) binding at least one of the plurality of covalently bound peptide second tags to an antibody (or another type of agent, for example a monoclonal antibody, a polyclonal antibody, a multivalent antibody, a chimeric antibody, a multispecific antibody, or an antibody fragment thereof; an aptamer; an affimer; a protein; a protein receptor; a protein receptor; a protein ligand; or a fusion protein, comprising, for example, an immunoglobulin fusion partner, a fusion partner that stabilizes a receptor or a ligand, or a fusion partner that provides a target for binding) conjugated to a second bead; and
iv) detecting the presence of the analyte by sensing an emission resulting from an interaction between the attached first bead and the bound second bead.

In an embodiment, a method for detecting the presence of, or determining the absence of, a specific analyte in a sample comprises:
i) mixing a tagged first agent and a tagged second agent into the sample, wherein:
   a) the tagged first agent is capable of binding with the specific analyte; and
   b) the tagged second agent comprises a plurality of covalently bound peptide second tags and is capable of separately binding with the specific analyte;
ii) capturing the tagged first agent with a first bead;
iii) binding the tagged second agent to a second bead; and
iv) detecting the presence of, or determining the absence of, the specific analyte by sensing an emission resulting from an interaction between the captured first bead and said bound second bead.

In an embodiment, a method of detecting an analyte in the presence of a plurality of non-analyte antibodies comprises:
i) mixing a first agent and a tagged second agent into a sample comprising the analyte and the plurality of non-analyte antibodies, wherein the tagged second agent comprises a plurality of covalently bound peptide second tags;
ii) binding the first agent directly to the analyte and binding the tagged second agent directly to the analyte; and
iii) introducing a first bead comprising a binder and a second bead comprising an anti-peptide agent to the bound analyte, wherein:
   a) the binder preferentially binds to the first agent over the non-analyte antibodies; and
   b) the anti-peptide agent is capable of binding to at least one of the covalently bound peptide second tags.
iv) detecting the presence of the analyte by sensing an emission resulting from an interaction between the attached first bead and the bound second bead.

In an embodiment, a method of detecting an analyte in the presence of a plurality of non-analyte antibodies comprises:
i) mixing a first agent and a tagged second agent into a sample comprising the analyte and the plurality of non-analyte antibodies, wherein the tagged second agent comprises a plurality of covalently bound peptide second tags;
ii) binding the first agent directly to the analyte and binding the tagged second agent directly to the analyte; and
iii) introducing a first bead comprising a binder and a second bead comprising an anti-peptide agent to the bound analyte, wherein:
   a) the binder preferentially binds to the first agent over the non-analyte antibodies; and
   b) the anti-peptide agent is capable of binding to at least one of the covalently bound peptide second tags.
iv) capturing the tagged first agent with the binder and binding at least one of the plurality of covalently bound peptide second tags to the anti-peptide agent conjugated to the second bead; and
v) detecting the presence of the analyte by sensing an emission resulting from an interaction between the attached first bead and the bound second bead.

In an embodiment, a method for detecting at least two different analytes in a sample comprises:
i) binding a donor antibody to a first analyte and separately binding a tagged acceptor antibody to the first analyte, the tagged acceptor antibody comprising a plurality of covalently bound peptide tags;
ii) binding a tagged further donor antibody to a second analyte and separately binding a further acceptor antibody to the second analyte;
iii) attaching the donor antibody to a binder conjugated on a donor bead and attaching the further donor antibody to a further binder conjugated on the donor bead;
iv) binding at least one of the plurality of covalently bound peptide tags to an anti-peptide agent conjugated to a first acceptor bead;
v) binding the further acceptor antibody to a second acceptor bead; and
vi) detecting the at least two different analytes by sensing the product of an interaction between the donor bead and the first and second acceptor beads.

In an embodiment, a method for detecting at least two different analytes in a sample comprises:
i) binding a donor agent to a first analyte and separately binding a tagged acceptor agent to the first analyte, the tagged acceptor agent comprising a plurality of covalently bound peptide tags;
ii) binding a tagged further donor agent to a second analyte and separately binding a further acceptor agent to the second analyte;
iii) attaching the donor agent to a binder conjugated on a donor bead and attaching the further donor agent to a further binder conjugated on the donor bead;
iv) binding at least one of the plurality of covalently bound peptide tags to an anti-peptide agent conjugated to a first acceptor bead;
v) binding the further acceptor agent to a second acceptor bead; and
vi) detecting the at least two different analytes by sensing the product of an interaction between the donor bead and the first and second acceptor beads.

In an embodiment, a method for detecting at least two epitopes of an analyte comprises:
i) binding a tagged donor agent to the analyte;
ii) attaching the tagged donor agent to a binder conjugated on a donor bead;
iii) binding a tagged first acceptor agent to a first epitope of the analyte and separately binding a second acceptor agent to a second epitope of the analyte, said tagged first acceptor agent comprising a plurality of covalently bound peptide first tags;
iv) binding at least one of the plurality of covalently bound peptide tags to an anti-peptide first agent conjugated to a first acceptor bead;
v) binding the second acceptor agent to a second acceptor bead; and
vi) detecting the at least two different analytes by sensing the product of an interaction between the donor bead and the two acceptor beads.

In an embodiment, a detection complex comprises:
i) one or more analyte complexes comprises:
   a) an analyte;
   b) a first agent comprising one or more first tags and bound to the analyte; and
   c) a second agent covalently bound to a plurality of peptide tags and separately bound to said analyte;
ii) a first bead having a plurality of binders conjugated thereon, wherein at least one of the one or more first tags present from the one or more analyte complexes is bound to one of the plurality of binders; and
iii) a second bead having a plurality of anti-peptide agents conjugated thereon, wherein at least one of the plurality of covalently bound peptide second tags from the one or more analyte complexes is bound to one of the plurality of anti-peptide agents.

In an embodiment, a multi-analyte detection complex comprises:
i) a first analyte complex, comprising:
   a) a first analyte;
   b) a tagged donor antibody bound to the first analyte, the tagged donor antibody comprising one or more donor tags; and
   c) a tagged acceptor antibody bound to the first analyte, the tagged acceptor antibody comprising a plurality of covalently bound peptide tags;
ii) a second analyte complex, comprising:
   a) an second analyte;
   b) a further donor antibody bound to the second analyte; and
   c) a further acceptor antibody bound to the second analyte;
iii) a donor bead having a plurality of binders conjugated thereon, at least one of the one or more first tags present from each of the first and second analyte complexes bound to one of the plurality of binders, and the a further donor antibody bound to one of the plurality of binders;
iv) a first acceptor bead having a plurality of anti-peptide antibodies conjugated thereon, at least one of the plurality of covalently bound peptide second tags bound to one of the plurality of anti-peptide antibodies; and
v) a second acceptor bead bound to the further acceptor antibody.

In an embodiment, a multi-analyte detection complex comprises:
i) a first analyte complex, comprising:
   a) a first analyte;
   b) a tagged donor agent bound to the first analyte, the tagged donor agent comprising one or more donor tags; and
   c) a tagged acceptor agent bound to the first analyte, the tagged acceptor agent comprising a plurality of covalently bound peptide tags;
ii) a second analyte complex, comprising:
   a) an second analyte;
   b) a further donor agent bound to the second analyte; and
   c) a further acceptor agent bound to the second analyte;
iii) a donor bead having a plurality of binders conjugated thereon, at least one of the one or more first tags present from each of the first and second analyte complexes bound to one of the plurality of binders, and the a further donor agent bound to one of the plurality of binders;
iv) a first acceptor bead having a plurality of anti-peptide agents conjugated thereon, at least one of the plurality of covalently bound peptide second tags bound to one of the plurality of anti-peptide agents; and
v) a second acceptor bead bound to the further acceptor agent.

In an embodiment, a multi-epitope detection complex comprises:
i) an analyte complex, comprising:
   a) an analyte;
   b) a tagged donor agent bound to the analyte, the tagged donor agent comprising one or more donor tags;
   c) a tagged acceptor agent bound to a first epitope of the analyte, the tagged acceptor agent comprising a plurality of covalently bound peptide tags; and
   d) a further acceptor agent bound to a second epitope of the analyte;
ii) a donor bead having a plurality of binders conjugated thereon, wherein at least one of the one or more donor tags is bound to one of the plurality of binders;
iii) a first acceptor bead having a plurality of anti-peptide agents conjugated thereon, at least one of the plurality of covalently bound peptide second tags bound to one of the plurality of anti-peptide agents; and
iv) a second acceptor bead bound to the further acceptor agent.

In an embodiment, a method of detecting an analyte in a sample comprises:
i) forming a plurality of detection complexes dispersed in the sample, wherein the first beads are capable of a chemical transfer interactions with the second beads;
ii) irradiating the donor beads in at least a portion of the detection complexes with light at an appropriate wavelength to trigger excitation of at least a portion of the acceptor beads in the detection complexes; and
iii) detecting light emitted by the acceptor beads.

In certain embodiments, one or more than one (including for instance all) of the following further embodiments may comprise each of the other embodiments or parts thereof.

In a further embodiment, the system, kit, method, or detection complex of any one of the above embodiments and any one or more of the further embodiments herein, wherein the first agent is a monoclonal antibody

In a further embodiment, the system, kit, method, or detection complex of any one of the above embodiments and any one or more of the further embodiments herein, wherein the first agent is a polyclonal antibody.

In a further embodiment, the system, kit, method, or detection complex of any one of the above embodiments and any one or more of the further embodiments herein, wherein the first agent is a multivalent antibody.

In a further embodiment, the system, kit, method, or detection complex of any one of the above embodiments and any one or more of the further embodiments herein, wherein the first agent is a chimeric antibody.

In a further embodiment, the system, kit, method, or detection complex of any one of the above embodiments and any one or more of the further embodiments herein, wherein the first agent is a multispecific antibody.

In a further embodiment, the system, kit, method, or detection complex of any one of the above embodiments and any one or more of the further embodiments herein, wherein the first agent is an antibody fragment.

In a further embodiment, the system, kit, method, or detection complex of any one of the above embodiments and any one or more of the further embodiments herein, wherein the first agent is an aptamer.

In a further embodiment, the system, kit, method, or detection complex of any one of the above embodiments and any one or more of the further embodiments herein, wherein the first agent is an affimer.

In a further embodiment, the system, kit, method, or detection complex of any one of the above embodiments and any one or more of the further embodiments herein, wherein the first agent is a protein.

In a further embodiment, the system, kit, method, or detection complex of any one of the above embodiments and any one or more of the further embodiments herein, wherein the first agent is a protein receptor.

In a further embodiment, the system, kit, method, or detection complex of any one of the above embodiments and any one or more of the further embodiments herein, wherein the first agent is a protein receptor.

In a further embodiment, the system, kit, method, or detection complex of any one of the above embodiments and any one or more of the further embodiments herein, wherein the first agent is a protein ligand.

In a further embodiment, the system, kit, method, or detection complex of any one of the above embodiments and any one or more of the further embodiments herein, wherein the first agent is a fusion protein.

In a further embodiment, the system, kit, method, or detection complex of any one of the above embodiments and any one or more of the further embodiments herein, wherein the first agent is an immunoglobulin fusion partner.

In a further embodiment, the system, kit, method, or detection complex of any one of the above embodiments and any one or more of the further embodiments herein, wherein the first agent is a fusion partner that stabilizes a receptor or a ligand.

In a further embodiment, the system, kit, method, or detection complex of any one of the above embodiments and any one or more of the further embodiments herein, wherein the first agent is a fusion partner that provides a target for binding.

In a further embodiment, the system, kit, method, or detection complex of any one of the above embodiments and any one or more of the further embodiments herein, wherein the second agent is a monoclonal antibody

In a further embodiment, the system, kit, method, or detection complex of any one of the above embodiments and any one or more of the further embodiments herein, wherein the second agent is a polyclonal antibody.

In a further embodiment, the system, kit, method, or detection complex of any one of the above embodiments and any one or more of the further embodiments herein, wherein the second agent is a multivalent antibody.

In a further embodiment, the system, kit, method, or detection complex of any one of the above embodiments and any one or more of the further embodiments herein, wherein the second agent is a chimeric antibody.

In a further embodiment, the system, kit, method, or detection complex of any one of the above embodiments and any one or more of the further embodiments herein, wherein the second agent is a multispecific antibody.

In a further embodiment, the system, kit, method, or detection complex of any one of the above embodiments and any one or more of the further embodiments herein, wherein the second agent is an antibody fragment.

In a further embodiment, the system, kit, method, or detection complex of any one of the above embodiments and any one or more of the further embodiments herein, wherein the second agent is an aptamer.

In a further embodiment, the system, kit, method, or detection complex of any one of the above embodiments and any one or more of the further embodiments herein, wherein the second agent is an affimer.

In a further embodiment, the system, kit, method, or detection complex of any one of the above embodiments and any one or more of the further embodiments herein, wherein the second agent is a protein.

In a further embodiment, the system, kit, method, or detection complex of any one of the above embodiments and any one or more of the further embodiments herein, wherein the second agent is a protein receptor.

In a further embodiment, the system, kit, method, or detection complex of any one of the above embodiments and any one or more of the further embodiments herein, wherein the second agent is a protein receptor.

In a further embodiment, the system, kit, method, or detection complex of any one of the above embodiments and any one or more of the further embodiments herein, wherein the second agent is a protein ligand.

In a further embodiment, the system, kit, method, or detection complex of any one of the above embodiments and any one or more of the further embodiments herein, wherein the second agent is a fusion protein.

In a further embodiment, the system, kit, method, or detection complex of any one of the above embodiments and any one or more of the further embodiments herein, wherein the second agent is an immunoglobulin fusion partner.

In a further embodiment, the system, kit, method, or detection complex of any one of the above embodiments and any one or more of the further embodiments herein, wherein the second agent is a fusion partner that stabilizes a receptor or a ligand.

In a further embodiment, the system, kit, method, or detection complex of any one of the above embodiments and any one or more of the further embodiments herein, wherein the second agent is a fusion partner that provides a target for binding.

In a further embodiment, the system, kit, method, or detection complex of any one of the above embodiments and any one or more of the further embodiments herein, wherein the third agent is a monoclonal antibody

In a further embodiment, the system, kit, method, or detection complex of any one of the above embodiments and any one or more of the further embodiments herein, wherein the third agent is a polyclonal antibody.

In a further embodiment, the system, kit, method, or detection complex of any one of the above embodiments and any one or more of the further embodiments herein, wherein the third agent is a multivalent antibody.

In a further embodiment, the system, kit, method, or detection complex of any one of the above embodiments and any one or more of the further embodiments herein, wherein the third agent is a chimeric antibody.

In a further embodiment, the system, kit, method, or detection complex of any one of the above embodiments and any one or more of the further embodiments herein, wherein the third agent is a multispecific antibody.

In a further embodiment, the system, kit, method, or detection complex of any one of the above embodiments and any one or more of the further embodiments herein, wherein the third agent is an antibody fragment.

In a further embodiment, the system, kit, method, or detection complex of any one of the above embodiments and any one or more of the further embodiments herein, wherein the third agent is an aptamer.

In a further embodiment, the system, kit, method, or detection complex of any one of the above embodiments and any one or more of the further embodiments herein, wherein the third agent is an affimer.

In a further embodiment, the system, kit, method, or detection complex of any one of the above embodiments and any one or more of the further embodiments herein, wherein the third agent is a protein.

In a further embodiment, the system, kit, method, or detection complex of any one of the above embodiments and any one or more of the further embodiments herein, wherein the third agent is a protein receptor.

In a further embodiment, the system, kit, method, or detection complex of any one of the above embodiments and any one or more of the further embodiments herein, wherein the third agent is a protein receptor.

In a further embodiment, the system, kit, method, or detection complex of any one of the above embodiments and any one or more of the further embodiments herein, wherein the third agent is a protein ligand.

In a further embodiment, the system, kit, method, or detection complex of any one of the above embodiments and any one or more of the further embodiments herein, wherein the third agent is a fusion protein.

In a further embodiment, the system, kit, method, or detection complex of any one of the above embodiments and any one or more of the further embodiments herein, wherein the third agent is an immunoglobulin fusion partner.

In a further embodiment, the system, kit, method, or detection complex of any one of the above embodiments and any one or more of the further embodiments herein, wherein the third agent is a fusion partner that stabilizes a receptor or a ligand.

In a further embodiment, the system, kit, method, or detection complex of any one of the above embodiments and any one or more of the further embodiments herein, wherein the third agent is a fusion partner that provides a target for binding.

In a further embodiment, the system, kit, method, or detection complex of any one of the above embodiments and any one or more of the further embodiments herein, wherein the fourth agent is a monoclonal antibody

In a further embodiment, the system, kit, method, or detection complex of any one of the above embodiments and any one or more of the further embodiments herein, wherein the fourth agent is a polyclonal antibody.

In a further embodiment, the system, kit, method, or detection complex of any one of the above embodiments and any one or more of the further embodiments herein, wherein the fourth agent is a multivalent antibody.

In a further embodiment, the system, kit, method, or detection complex of any one of the above embodiments and any one or more of the further embodiments herein, wherein the fourth agent is a chimeric antibody.

In a further embodiment, the system, kit, method, or detection complex of any one of the above embodiments and any one or more of the further embodiments herein, wherein the fourth agent is a multispecific antibody.

In a further embodiment, the system, kit, method, or detection complex of any one of the above embodiments and any one or more of the further embodiments herein, wherein the fourth agent is an antibody fragment.

In a further embodiment, the system, kit, method, or detection complex of any one of the above embodiments and any one or more of the further embodiments herein, wherein the fourth agent is an aptamer.

In a further embodiment, the system, kit, method, or detection complex of any one of the above embodiments and any one or more of the further embodiments herein, wherein the fourth agent is an affimer.

In a further embodiment, the system, kit, method, or detection complex of any one of the above embodiments and any one or more of the further embodiments herein, wherein the fourth agent is a protein.

In a further embodiment, the system, kit, method, or detection complex of any one of the above embodiments and any one or more of the further embodiments herein, wherein the fourth agent is a protein receptor.

In a further embodiment, the system, kit, method, or detection complex of any one of the above embodiments and any one or more of the further embodiments herein, wherein the fourth agent is a protein receptor.

In a further embodiment, the system, kit, method, or detection complex of any one of the above embodiments and any one or more of the further embodiments herein, wherein the fourth agent is a protein ligand.

In a further embodiment, the system, kit, method, or detection complex of any one of the above embodiments and any one or more of the further embodiments herein, wherein the fourth agent is a fusion protein.

In a further embodiment, the system, kit, method, or detection complex of any one of the above embodiments and any one or more of the further embodiments herein, wherein the fourth agent is an immunoglobulin fusion partner.

In a further embodiment, the system, kit, method, or detection complex of any one of the above embodiments and any one or more of the further embodiments herein, wherein the fourth agent is a fusion partner that stabilizes a receptor or a ligand.

In a further embodiment, the system, kit, method, or detection complex of any one of the above embodiments and any one or more of the further embodiments herein, wherein the fourth agent is a fusion partner that provides a target for binding.

In a further embodiment, the system, kit, method, or detection complex of any one of the above embodiments and any one or more of the further embodiments herein, wherein the anti-peptide agent is a monoclonal antibody

In a further embodiment, the system, kit, method, or detection complex of any one of the above embodiments and any one or more of the further embodiments herein, wherein the anti-peptide agent is a polyclonal antibody.

In a further embodiment, the system, kit, method, or detection complex of any one of the above embodiments and any one or more of the further embodiments herein, wherein the anti-peptide agent is a multivalent antibody.

In a further embodiment, the system, kit, method, or detection complex of any one of the above embodiments and any one or more of the further embodiments herein, wherein the anti-peptide agent is a chimeric antibody.

In a further embodiment, the system, kit, method, or detection complex of any one of the above embodiments and any one or more of the further embodiments herein, wherein the anti-peptide agent is a multispecific antibody.

In a further embodiment, the system, kit, method, or detection complex of any one of the above embodiments and any one or more of the further embodiments herein, wherein the anti-peptide agent is an antibody fragment.

In a further embodiment, the system, kit, method, or detection complex of any one of the above embodiments and any one or more of the further embodiments herein, wherein the anti-peptide agent is an aptamer.

In a further embodiment, the system, kit, method, or detection complex of any one of the above embodiments and any one or more of the further embodiments herein, wherein the anti-peptide agent is an affimer.

In a further embodiment, the system, kit, method, or detection complex of any one of the above embodiments and any one or more of the further embodiments herein, wherein the anti-peptide agent is a protein.

In a further embodiment, the system, kit, method, or detection complex of any one of the above embodiments and any one or more of the further embodiments herein, wherein the anti-peptide agent is a protein receptor.

In a further embodiment, the system, kit, method, or detection complex of any one of the above embodiments and any one or more of the further embodiments herein, wherein the anti-peptide agent is a protein receptor.

In a further embodiment, the system, kit, method, or detection complex of any one of the above embodiments and any one or more of the further embodiments herein, wherein the anti-peptide agent is a protein ligand.

In a further embodiment, the system, kit, method, or detection complex of any one of the above embodiments and any one or more of the further embodiments herein, wherein the anti-peptide agent is a fusion protein.

In a further embodiment, the system, kit, method, or detection complex of any one of the above embodiments and any one or more of the further embodiments herein, wherein the anti-peptide agent is an immunoglobulin fusion partner.

In a further embodiment, the system, kit, method, or detection complex of any one of the above embodiments and any one or more of the further embodiments herein, wherein the anti-peptide agent is a fusion partner that stabilizes a receptor or a ligand.

In a further embodiment, the system, kit, method, or detection complex of any one of the above embodiments and any one or more of the further embodiments herein, wherein the anti-peptide agent is a fusion partner that provides a target for binding.

In a further embodiment, the system, kit, method, or detection complex of any one of the above embodiments and any one or more of the further embodiments herein, wherein the binder is a monoclonal antibody

In a further embodiment, the system, kit, method, or detection complex of any one of the above embodiments and any one or more of the further embodiments herein, wherein the binder is a polyclonal antibody.

In a further embodiment, the system, kit, method, or detection complex of any one of the above embodiments and any one or more of the further embodiments herein, wherein the binder is a multivalent antibody.

In a further embodiment, the system, kit, method, or detection complex of any one of the above embodiments and any one or more of the further embodiments herein, wherein the binder is a chimeric antibody.

In a further embodiment, the system, kit, method, or detection complex of any one of the above embodiments and any one or more of the further embodiments herein, wherein the binder is a multispecific antibody.

In a further embodiment, the system, kit, method, or detection complex of any one of the above embodiments and any one or more of the further embodiments herein, wherein the binder is an antibody fragment.

In a further embodiment, the system, kit, method, or detection complex of any one of the above embodiments and any one or more of the further embodiments herein, wherein the binder is an aptamer.

In a further embodiment, the system, kit, method, or detection complex of any one of the above embodiments and any one or more of the further embodiments herein, wherein the binder is an affimer.

In a further embodiment, the system, kit, method, or detection complex of any one of the above embodiments and any one or more of the further embodiments herein, wherein the binder is a protein.

In a further embodiment, the system, kit, method, or detection complex of any one of the above embodiments and any one or more of the further embodiments herein, wherein the binder is a protein receptor.

In a further embodiment, the system, kit, method, or detection complex of any one of the above embodiments and any one or more of the further embodiments herein, wherein the binder is a protein receptor.

In a further embodiment, the system, kit, method, or detection complex of any one of the above embodiments and any one or more of the further embodiments herein, wherein the binder is a protein ligand.

In a further embodiment, the system, kit, method, or detection complex of any one of the above embodiments and any one or more of the further embodiments herein, wherein the binder is a fusion protein.

In a further embodiment, the system, kit, method, or detection complex of any one of the above embodiments and any one or more of the further embodiments herein, wherein the binder is an immunoglobulin fusion partner.

In a further embodiment, the system, kit, method, or detection complex of any one of the above embodiments and any one or more of the further embodiments herein, wherein the binder is a fusion partner that stabilizes a receptor or a ligand.

In a further embodiment, the system, kit, method, or detection complex of any one of the above embodiments and any one or more of the further embodiments herein, wherein the binder is a fusion partner that provides a target for binding.

In a further embodiment, the system, kit, method, or detection complex of any one of the above embodiments and any one or more of the further embodiments herein, wherein the donor agent is a monoclonal antibody

In a further embodiment, the system, kit, method, or detection complex of any one of the above embodiments and any one or more of the further embodiments herein, wherein the donor agent is a polyclonal antibody.

In a further embodiment, the system, kit, method, or detection complex of any one of the above embodiments and any one or more of the further embodiments herein, wherein the donor agent is a multivalent antibody.

In a further embodiment, the system, kit, method, or detection complex of any one of the above embodiments and any one or more of the further embodiments herein, wherein the donor agent is a chimeric antibody.

In a further embodiment, the system, kit, method, or detection complex of any one of the above embodiments and any one or more of the further embodiments herein, wherein the donor agent is a multispecific antibody.

In a further embodiment, the system, kit, method, or detection complex of any one of the above embodiments and any one or more of the further embodiments herein, wherein the donor agent is an antibody fragment.

In a further embodiment, the system, kit, method, or detection complex of any one of the above embodiments and any one or more of the further embodiments herein, wherein the donor agent is an aptamer.

In a further embodiment, the system, kit, method, or detection complex of any one of the above embodiments and any one or more of the further embodiments herein, wherein the donor agent is an affimer.

In a further embodiment, the system, kit, method, or detection complex of any one of the above embodiments and any one or more of the further embodiments herein, wherein the donor agent is a protein.

In a further embodiment, the system, kit, method, or detection complex of any one of the above embodiments and any one or more of the further embodiments herein, wherein the donor agent is a protein receptor.

In a further embodiment, the system, kit, method, or detection complex of any one of the above embodiments and any one or more of the further embodiments herein, wherein the donor agent is a protein receptor.

In a further embodiment, the system, kit, method, or detection complex of any one of the above embodiments and any one or more of the further embodiments herein, wherein the donor agent is a protein ligand.

In a further embodiment, the system, kit, method, or detection complex of any one of the above embodiments and any one or more of the further embodiments herein, wherein the donor agent is a fusion protein.

In a further embodiment, the system, kit, method, or detection complex of any one of the above embodiments and any one or more of the further embodiments herein, wherein the donor agent is an immunoglobulin fusion partner.

In a further embodiment, the system, kit, method, or detection complex of any one of the above embodiments and any one or more of the further embodiments herein, wherein the donor agent is a fusion partner that stabilizes a receptor or a ligand.

In a further embodiment, the system, kit, method, or detection complex of any one of the above embodiments and any one or more of the further embodiments herein, wherein the donor agent is a fusion partner that provides a target for binding.

In a further embodiment, the system, kit, method, or detection complex of any one of the above embodiments and any one or more of the further embodiments herein, wherein the acceptor agent is a monoclonal antibody

In a further embodiment, the system, kit, method, or detection complex of any one of the above embodiments and any one or more of the further embodiments herein, wherein the acceptor agent is a polyclonal antibody.

In a further embodiment, the system, kit, method, or detection complex of any one of the above embodiments and any one or more of the further embodiments herein, wherein the acceptor agent is a multivalent antibody.

In a further embodiment, the system, kit, method, or detection complex of any one of the above embodiments and any one or more of the further embodiments herein, wherein the acceptor agent is a chimeric antibody.

In a further embodiment, the system, kit, method, or detection complex of any one of the above embodiments and any one or more of the further embodiments herein, wherein the acceptor agent is a multispecific antibody.

In a further embodiment, the system, kit, method, or detection complex of any one of the above embodiments and any one or more of the further embodiments herein, wherein the acceptor agent is an antibody fragment.

In a further embodiment, the system, kit, method, or detection complex of any one of the above embodiments and any one or more of the further embodiments herein, wherein the acceptor agent is an aptamer.

In a further embodiment, the system, kit, method, or detection complex of any one of the above embodiments and any one or more of the further embodiments herein, wherein the acceptor agent is an affimer.

In a further embodiment, the system, kit, method, or detection complex of any one of the above embodiments and any one or more of the further embodiments herein, wherein the acceptor agent is a protein.

In a further embodiment, the system, kit, method, or detection complex of any one of the above embodiments and any one or more of the further embodiments herein, wherein the acceptor agent is a protein receptor.

In a further embodiment, the system, kit, method, or detection complex of any one of the above embodiments and any one or more of the further embodiments herein, wherein the acceptor agent is a protein receptor.

In a further embodiment, the system, kit, method, or detection complex of any one of the above embodiments and any one or more of the further embodiments herein, wherein the acceptor agent is a protein ligand.

In a further embodiment, the system, kit, method, or detection complex of any one of the above embodiments and any one or more of the further embodiments herein, wherein the acceptor agent is a fusion protein.

In a further embodiment, the system, kit, method, or detection complex of any one of the above embodiments and any one or more of the further embodiments herein, wherein the acceptor agent is an immunoglobulin fusion partner.

In a further embodiment, the system, kit, method, or detection complex of any one of the above embodiments and any one or more of the further embodiments herein, wherein the acceptor agent is a fusion partner that stabilizes a receptor or a ligand.

In a further embodiment, the system, kit, method, or detection complex of any one of the above embodiments and any one or more of the further embodiments herein, wherein the acceptor agent is a fusion partner that provides a target for binding.

In a further embodiment, the system, kit, method, or detection complex of any one of the above embodiments and any one or more of the further embodiments herein, wherein the tagged first agent is capable of binding to a first epitope of the analyte and the second agent is capable of binding to a second epitope of the analyte.

In a further embodiment, the system, kit, method, or detection complex of any one of the above embodiments and any one or more of the further embodiments herein, wherein the first epitope of the analyte and the second epitope of the analyte are the same type of epitope.

In a further embodiment, the system, kit, method, or detection complex of any one of the above embodiments and any one or more of the further embodiments herein, wherein the first epitope of the analyte and the second epitope of the analyte are two different types of epitope.

In a further embodiment, the system, kit, method, or detection complex of any one of the above embodiments and any one or more of the further embodiments herein, wherein the first epitope of the analyte and/or the second epitope of the analyte is a phospho-epitope.

In a further embodiment, the system, kit, method, or detection complex of any one of the above embodiments and any one or more of the further embodiments herein, wherein the at least one tag is conjugated to the first agent.

In a further embodiment, the system, kit, method, or detection complex of any one of the above embodiments and any one or more of the further embodiments herein, wherein the at least one tag is covalently bound to the first agent.

In a further embodiment, the system, kit, method, or detection complex of any one of the above embodiments and any one or more of the further embodiments herein, wherein the at least one tag is a ligand.

In a further embodiment, the system, kit, method, or detection complex of any one of the above embodiments and any one or more of the further embodiments herein, wherein the first agent comprises a plurality of tags.

In a further embodiment, the system, kit, method, or detection complex of any one of the above embodiments and any one or more of the further embodiments herein, wherein the first agent comprises in the range of between 1-15 tags.

In a further embodiment, the system, kit, method, or detection complex of any one of the above embodiments and any one or more of the further embodiments herein, wherein the first agent comprises in the range of between 2-9 tags.

In a further embodiment, the system, kit, method, or detection complex of any one of the above embodiments and any one or more of the further embodiments herein, wherein the first agent comprises in the range of between 5-8 tags.

In a further embodiment, the system, kit, method, or detection complex of any one of the above embodiments and any one or more of the further embodiments herein, wherein at least one of the plurality of tags of the tagged first agent is a ligand.

In a further embodiment, the system, kit, method, or detection complex of any one of the above embodiments and any one or more of the further embodiments herein, wherein the ligand is biotin.

In a further embodiment, the system, kit, method, or detection complex of any one of the above embodiments and any one or more of the further embodiments herein, wherein the ligand is a peptide tag.

In a further embodiment, the system, kit, method, or detection complex of any one of the above embodiments and any one or more of the further embodiments herein, wherein the binder selectively binds to the at least one tag of the tagged first agent.

In a further embodiment, the system, kit, method, or detection complex of any one of the above embodiments and any one or more of the further embodiments herein, wherein the binder is a further antibody.

In a further embodiment, the system, kit, method, or detection complex of any one of the above embodiments and any one or more of the further embodiments herein, wherein the binder is a further agent.

In a further embodiment, the system, kit, method, or detection complex of any one of the above embodiments and any one or more of the further embodiments herein, wherein the binder is streptavidin.

In a further embodiment, the system, kit, method, or detection complex of any one of the above embodiments and any one or more of the further embodiments herein, wherein the further antibody has an affinity for the at least one tag.

In a further embodiment, the system, kit, method, or detection complex of any one of the above embodiments and any one or more of the further embodiments herein, wherein the further antibody selectively binds to the at least one tag.

In a further embodiment, the system, kit, method, or detection complex of any one of the above embodiments and any one or more of the further embodiments herein, wherein the further agent has an affinity for the at least one tag.

In a further embodiment, the system, kit, method, or detection complex of any one of the above embodiments and any one or more of the further embodiments herein, wherein the further agent selectively binds to the at least one tag.

In a further embodiment, the system, kit, method, or detection complex of any one of the above embodiments and any one or more of the further embodiments herein, wherein the second agent is covalently bound to in the range of between 1-15 peptide tags.

In a further embodiment, the system, kit, method, or detection complex of any one of the above embodiments and any one or more of the further embodiments herein, wherein the second agent is covalently bound to in the range of between 2-9 peptide tags.

In a further embodiment, the system, kit, method, or detection complex of any one of the above embodiments and any one or more of the further embodiments herein, wherein the second agent is covalently bound to in the range of between 5-8 peptide tags.

In a further embodiment, the system, kit, method, or detection complex of any one of the above embodiments and any one or more of the further embodiments herein, wherein at least one of the covalently bound peptide tags comprise no more than 30 amino acids.

In a further embodiment, the system, kit, method, or detection complex of any one of the above embodiments and any one or more of the further embodiments herein, wherein at least one of the covalently bound peptide tags comprise no more than 25 amino acids.

In a further embodiment, the system, kit, method, or detection complex of any one of the above embodiments and any one or more of the further embodiments herein, wherein at least one of the covalently bound peptide tags comprise no more than 10 amino acids.

In a further embodiment, the system, kit, method, or detection complex of any one of the above embodiments and any one or more of the further embodiments herein, wherein at least one of the covalently bound peptide tags comprise no more than 8 amino acids.

In a further embodiment, the system, kit, method, or detection complex of any one of the above embodiments and any one or more of the further embodiments herein, wherein at least one of the plurality of covalently bound peptide tags comprises in the range of between 5-15 amino acids.

In a further embodiment, the system, kit, method, or detection complex of any one of the above embodiments and any one or more of the further embodiments herein, wherein at least one of the plurality of covalently bound peptide tags comprises in the range of between 6-12 amino acids.

In a further embodiment, the system, kit, method, or detection complex of any one of the above embodiments and any one or more of the further embodiments herein, wherein at least one of the plurality of covalently bound peptide tags comprises in the range of between 7-10 amino acids.

In a further embodiment, the system, kit, method, or detection complex of any one of the above embodiments and any one or more of the further embodiments herein, wherein at least one of the plurality of covalently bound peptide tags comprises in the range of between 8-9 amino acids.

In a further embodiment, the system, kit, method, or detection complex of any one of the above embodiments and any one or more of the further embodiments herein, wherein at least one of the plurality of covalently bound peptide tags comprises the amino acid sequence DYKDDDDK (SEQ ID NO: 1).

In a further embodiment, the system, kit, method, or detection complex of any one of the above embodiments and any one or more of the further embodiments herein, wherein at least one of the plurality of covalently bound peptide tags consists of an amino acid sequence DYKDDDDK (SEQ ID NO: 1).

In a further embodiment, the system, kit, method, or detection complex of any one of the above embodiments and any one or more of the further embodiments herein, wherein at least one of the plurality of covalently bound peptide tags comprises the amino acid sequence CDYKDDDDK (SEQ ID NO: 8).

In a further embodiment, the system, kit, method, or detection complex of any one of the above embodiments and any one or more of the further embodiments herein, wherein at least one of the plurality of covalently bound peptide tags consists of an amino acid sequence CDYKDDDDK (SEQ ID NO: 8).

In a further embodiment, the system, kit, method, or detection complex of any one of the above embodiments and any one or more of the further embodiments herein, wherein the first bead and/or the second bead has a diameter in the range of between 250-350 nm.

In a further embodiment, the system, kit, method, or detection complex of any one of the above embodiments and any one or more of the further embodiments herein, wherein the first bead and/or the second bead is a latex bead.

In a further embodiment, the system, kit, method, or detection complex of any one of the above embodiments and any one or more of the further embodiments herein, wherein the first bead and the second bead are capable of interacting via a chemical transfer interaction.

In a further embodiment, the system, kit, method, or detection complex of any one of the above embodiments and any one or more of the further embodiments herein, wherein the first bead comprises a photosensitizer capable in its excited state of generating singlet oxygen.

In a further embodiment, the system, kit, method, or detection complex of any one of the above embodiments and any one or more of the further embodiments herein, wherein the first bead is a donor bead and the second bead is an acceptor bead.

In a further embodiment, the system, kit, method, or detection complex of any one of the above embodiments and any one or more of the further embodiments herein, wherein the donor bead emits singlet oxygen when stimulated by light.

In a further embodiment, the system, kit, method, or detection complex of any one of the above embodiments and any one or more of the further embodiments herein, wherein the donor bead emits singlet oxygen when stimulated by light at a wavelength in the range of between 620-700 nm.

In a further embodiment, the system, kit, method, or detection complex of any one of the above embodiments and any one or more of the further embodiments herein, wherein the donor bead emits singlet oxygen when stimulated by light at a wavelength in the range of between 650-700 nm.

In a further embodiment, the system, kit, method, or detection complex of any one of the above embodiments and any one or more of the further embodiments herein, wherein the donor bead emits singlet oxygen when stimulated by light at a wavelength of 680 nm.

In a further embodiment, the system, kit, method, or detection complex of any one of the above embodiments and any one or more of the further embodiments herein, wherein the acceptor bead comprises rubrene, europium, a europium chelate, samarium, or terbium.

In a further embodiment, the system, kit, method, or detection complex of any one of the above embodiments and any one or more of the further embodiments herein, wherein the chemical transfer interaction with the donor bead is an emission of singlet oxygen from the donor bead.

In a further embodiment, the system, kit, method, or detection complex of any one of the above embodiments and any one or more of the further embodiments herein, wherein the acceptor bead comprises a photoactive indicator precursor that reacts with singlet oxygen forming a photoactive indicator;

In a further embodiment, the system, kit, method, or detection complex of any one of the above embodiments and any one or more of the further embodiments herein, wherein the irradiated photoactive indicator on the acceptor bead emits fluorescence at a wavelength in the range of between 500-625 nm.

In a further embodiment, the system, kit, method, or detection complex of any one of the above embodiments and any one or more of the further embodiments herein, wherein the irradiated photoactive indicator on the acceptor bead emits fluorescence at a wavelength in the range of between 525-575 nm.

In a further embodiment, the system, kit, method, or detection complex of any one of the above embodiments and any one or more of the further embodiments herein, wherein the irradiated photoactive indicator on the acceptor bead emits fluorescence at a wavelength of 545 nm.

In a further embodiment, the system, kit, method, or detection complex of any one of the above embodiments and any one or more of the further embodiments herein, wherein the irradiated photoactive indicator on the acceptor bead emits fluorescence at a wavelength in the range of between 600-625 nm.

In a further embodiment, the system, kit, method, or detection complex of any one of the above embodiments and any one or more of the further embodiments herein, wherein the irradiated photoactive indicator on the acceptor bead emits fluorescence at a wavelength of 615 nm.

In a further embodiment, the system, kit, method, or detection complex of any one of the above embodiments and any one or more of the further embodiments herein, wherein the emitted fluorescence has a peak in the range 520 to 620 nm.

In a further embodiment, the system, kit, method, or detection complex of any one of the above embodiments and any one or more of the further embodiments herein, wherein the emitted fluorescence has a peak at 615 nm.

In a further embodiment, the system, kit, method, or detection complex of any one of the above embodiments and any one or more of the further embodiments herein, wherein the donor bead and the acceptor bead are capable of interacting via a chemical transfer interaction when the two beads are disposed within 200 nm of one another.

In a further embodiment, the system, kit, method, or detection complex of any one of the above embodiments and any one or more of the further embodiments herein, wherein at least one of the plurality of covalently bound peptide tags comprises an epitope of the anti-peptide agent.

In a further embodiment, the system, kit, method, or detection complex of any one of the above embodiments and any one or more of the further embodiments herein, further comprising exciting said photosensitizer by irradiation with light to produce singlet oxygen that reacts with said photoactive indicator precursor to produce said photoactive indicator; and irradiating said photoactive indicator and measuring the fluorescence emitted by said photoactive indicator.

In a further embodiment, the system, kit, method, or detection complex of any one of the above embodiments and any one or more of the further embodiments herein, wherein the emission is fluorescence and is emitted from the bound second bead.

In a further embodiment, the system, kit, method, or detection complex of any one of the above embodiments and any one or more of the further embodiments herein, wherein the tagged first agent binds to the analyte at a first epitope and the second agent binds to the analyte at a second epitope.

In a further embodiment, the system, kit, method, or detection complex of any one of the above embodiments and any one or more of the further embodiments herein, wherein the first epitope and the second epitope are distal.

In a further embodiment, the system, kit, method, or detection complex of any one of the above embodiments and any one or more of the further embodiments herein, wherein the first epitope and the second epitope are proximal.

In a further embodiment, the system, kit, method, or detection complex of any one of the above embodiments and any one or more of the further embodiments herein, wherein the first epitope and the second epitope are the same type of epitope.

In a further embodiment, the system, kit, method, or detection complex of any one of the above embodiments and any one or more of the further embodiments herein, wherein the first epitope and the second epitope are two different types of epitopes.

In a further embodiment, the system, kit, method, or detection complex of any one of the above embodiments and any one or more of the further embodiments herein, wherein the first epitope and/or the second epitope is a phospho-epitope.

In a further embodiment, the system, kit, method, or detection complex of any one of the above embodiments and any one or more of the further embodiments herein, wherein the one or more first tags comprises between 1-15 peptide tags.

In a further embodiment, the system, kit, method, or detection complex of any one of the above embodiments and any one or more of the further embodiments herein, wherein the one or more first tags comprises between 2-9 peptide tags.

In a further embodiment, the system, kit, method, or detection complex of any one of the above embodiments and any one or more of the further embodiments herein, wherein the one or more first tags comprises between 5-8 peptide tags.

In a further embodiment, the system, kit, method, or detection complex of any one of the above embodiments and any one or more of the further embodiments herein, wherein the one or more first tags comprises a plurality of biotin or a derivative thereof and the binder comprises streptavidin or a derivative thereof.

In a further embodiment, the system, kit, method, or detection complex of any one of the above embodiments and any one or more of the further embodiments herein, wherein the one or more first tags comprises a plurality of biotin and the binder comprises streptavidin.

In a further embodiment, the system, kit, method, or detection complex of any one of the above embodiments and any one or more of the further embodiments herein, wherein the one or more first tags comprises a plurality of covalently bound peptide first tags and the binder comprises a second anti-peptide agent.

In a further embodiment, the system, kit, method, or detection complex of any one of the above embodiments and any one or more of the further embodiments herein, wherein the binder is coated on the first bead.

In a further embodiment, the system, kit, method, or detection complex of any one of the above embodiments and any one or more of the further embodiments herein, wherein the anti-peptide agent is coated on the first bead.

In a further embodiment, the system, kit, method, or detection complex of any one of the above embodiments and any one or more of the further embodiments herein, wherein the binder is covalently bound on the first bead.

In a further embodiment, the system, kit, method, or detection complex of any one of the above embodiments and any one or more of the further embodiments herein, wherein the anti-peptide agent is covalently bound on the first bead.

In a further embodiment, the system, kit, method, or detection complex of any one of the above embodiments and any one or more of the further embodiments herein, wherein the binder is coated on the second bead.

In a further embodiment, the system, kit, method, or detection complex of any one of the above embodiments and any one or more of the further embodiments herein, wherein the anti-peptide agent is coated on the second bead.

In a further embodiment, the system, kit, method, or detection complex of any one of the above embodiments and any one or more of the further embodiments herein, wherein the binder is covalently bound on the second bead.

In a further embodiment, the system, kit, method, or detection complex of any one of the above embodiments and any one or more of the further embodiments herein, wherein the anti-peptide agent is covalently bound on the second bead.

In a further embodiment, the system, kit, method, or detection complex of any one of the above embodiments and any one or more of the further embodiments herein, wherein the analyte is provided in a sample.

In a further embodiment, the system, kit, method, or detection complex of any one of the above embodiments and any one or more of the further embodiments herein, wherein the sample, the tagged first agent, the tagged second agent, and the second bead are mixed together.

In a further embodiment, the system, kit, method, or detection complex of any one of the above embodiments and any one or more of the further embodiments herein, wherein the concentration of the anti-peptide agent is at least 2 times greater than the concentration of the tagged second agent in the mixture.

In a further embodiment, the system, kit, method, or detection complex of any one of the above embodiments and any one or more of the further embodiments herein, wherein the concentration of the anti-peptide agent is at least 5 times greater than the concentration of the tagged second agent in the mixture.

In a further embodiment, the system, kit, method, or detection complex of any one of the above embodiments and any one or more of the further embodiments herein, wherein the concentration of the anti-peptide agent is at least 10 times greater than the concentration of the tagged second agent in the mixture.

In a further embodiment, the system, kit, method, or detection complex of any one of the above embodiments and any one or more of the further embodiments herein, wherein the concentration of the anti-peptide agent is less than 500 ng/ml.

In a further embodiment, the system, kit, method, or detection complex of any one of the above embodiments and any one or more of the further embodiments herein, wherein the concentration of the anti-peptide agent is less than 250 ng/ml.

In a further embodiment, the system, kit, method, or detection complex of any one of the above embodiments and any one or more of the further embodiments herein, wherein the concentration of the anti-peptide agent is less than 50 ng/ml.

In a further embodiment, the system, kit, method, or detection complex of any one of the above embodiments and any one or more of the further embodiments herein, wherein the concentration of the analyte is in the range of 1 to 10 ng/ml.

In a further embodiment, the system, kit, method, or detection complex of any one of the above embodiments and any one or more of the further embodiments herein, wherein the concentration of the analyte is less than 1 ng/ml.

In a further embodiment, the system, kit, method, or detection complex of any one of the above embodiments and any one or more of the further embodiments herein, wherein the concentration of the analyte is in the range of 10 to 50 ng/ml.

In a further embodiment, the system, kit, method, or detection complex of any one of the above embodiments and any one or more of the further embodiments herein, wherein the concentration of the analyte is greater than 50 ng/ml.

In a further embodiment, the system, kit, method, or detection complex of any one of the above embodiments and any one or more of the further embodiments herein, wherein the first bead is added to the mixture and the resulting mixture is incubated for less than 30 minutes.

In a further embodiment, the system, kit, method, or detection complex of any one of the above embodiments and any one or more of the further embodiments herein, wherein the first bead is added to the mixture and the resulting mixture is incubated for less than one hour.

In a further embodiment, the system, kit, method, or detection complex of any one of the above embodiments and any one or more of the further embodiments herein, wherein the first bead is added to the mixture and the resulting mixture is incubated for more than one hour.

In a further embodiment, the system, kit, method, or detection complex of any one of the above embodiments and any one or more of the further embodiments herein, wherein the first bead is added to the mixture and the resulting mixture is incubated for up to two hours.

In a further embodiment, the system, kit, method, or detection complex of any one of the above embodiments and any one or more of the further embodiments herein, wherein the sample, the tagged first agent, the tagged second agent, and the second bead are incubated for approximately one hour.

In a further embodiment, the system, kit, method, or detection complex of any one of the above embodiments and any one or more of the further embodiments herein, wherein the sample, the tagged first agent, the tagged second agent, and the second bead are incubated for less than one hour.

In a further embodiment, the system, kit, method, or detection complex of any one of the above embodiments and any one or more of the further embodiments herein, wherein the sample, the tagged first agent, the tagged second agent, and the second bead are incubated for greater than one hour.

In a further embodiment, the system, kit, method, or detection complex of any one of the above embodiments and any one or more of the further embodiments herein, wherein the sample, the tagged first agent, the tagged second agent, and the second bead are incubated for up to two hours.

In a further embodiment, the system, kit, method, or detection complex of any one of the above embodiments and any one or more of the further embodiments herein, wherein the first bead is mixed with the incubated mixture and the resulting mixture is incubated for less than 30 minutes.

In a further embodiment, the system, kit, method, or detection complex of any one of the above embodiments and any one or more of the further embodiments herein, wherein the first bead is mixed with the incubated mixture and the resulting mixture is incubated for less than one hour.

In a further embodiment, the system, kit, method, or detection complex of any one of the above embodiments and any one or more of the further embodiments herein, wherein the first bead is mixed with the incubated mixture and the resulting mixture is incubated for approximately one hour.

In a further embodiment, the system, kit, method, or detection complex of any one of the above embodiments and any one or more of the further embodiments herein, wherein the first bead is mixed with the incubated mixture and the resulting mixture is incubated for more than one hour.

In a further embodiment, the system, kit, method, or detection complex of any one of the above embodiments and any one or more of the further embodiments herein, wherein the first bead is mixed with the incubated mixture and the resulting mixture is incubated for up to two hours.

In a further embodiment, the system, kit, method, or detection complex of any one of the above embodiments and any one or more of the further embodiments herein, wherein the emission is emitted from the bound second bead.

In a further embodiment, the system, kit, method, or detection complex of any one of the above embodiments and any one or more of the further embodiments herein, wherein the specific analyte is detected by irradiating the first bead with a first light and sensing a second light emitted by the second bead, the second light emitted in response to a chemical transfer interaction with the first bead.

In a further embodiment, the system, kit, method, or detection complex of any one of the above embodiments and any one or more of the further embodiments herein, wherein no second light is sensed upon irradiation of the first bead with a first light and further irradiation of the second bead.

In a further embodiment, the system, kit, method, or detection complex of any one of the above embodiments and any one or more of the further embodiments herein, wherein at least one of the plurality of covalently bound peptide tags binds to an anti-peptide agent conjugated to the second bead.

In a further embodiment, the system, kit, method, or detection complex of any one of the above embodiments and any one or more of the further embodiments herein, wherein the rate constant for binding of the binder to the first agent is at least 2 times larger than the rate constants for binding of the binder to any of the non-analyte antibodies.

In a further embodiment, the system, kit, method, or detection complex of any one of the above embodiments and any one or more of the further embodiments herein, wherein the rate constant for binding of the binder to the first agent is at least 5 times larger than the rate constants for binding of the binder to any of the non-analyte antibodies.

In a further embodiment, the system, kit, method, or detection complex of any one of the above embodiments and any one or more of the further embodiments herein, wherein the rate constant for binding of the binder to the first agent is at least 10 times larger than the rate constants for binding of the binder to any of the non-analyte antibodies.

In a further embodiment, the system, kit, method, or detection complex of any one of the above embodiments and any one or more of the further embodiments herein, wherein the rate constant for binding of the binder to the first agent is at least 100 times larger than the rate constants for binding of the binder to any of the non-analyte antibodies.

In a further embodiment, the system, kit, method, or detection complex of any one of the above embodiments and any one or more of the further embodiments herein, wherein the dissociation constant for binding of the binder to the first agent is at least 2 times smaller than the dissociation constants for binding of the binder to any of the non-analyte antibodies.

In a further embodiment, the system, kit, method, or detection complex of any one of the above embodiments and any one or more of the further embodiments herein, wherein the dissociation constant for binding of the binder to the first agent is at least 5 times smaller than the dissociation constants for binding of the binder to any of the non-analyte antibodies.

In a further embodiment, the system, kit, method, or detection complex of any one of the above embodiments and any one or more of the further embodiments herein, wherein the dissociation constant for binding of the binder to the first agent is at least 10 times smaller than the dissociation constants for binding of the binder to any of the non-analyte antibodies.

In a further embodiment, the system, kit, method, or detection complex of any one of the above embodiments and any one or more of the further embodiments herein, wherein the dissociation constant for binding of the binder to the first agent is at least 100 times smaller than the dissociation constants for binding of the binder to any of the non-analyte antibodies.

In a further embodiment, the system, kit, method, or detection complex of any one of the above embodiments and any one or more of the further embodiments herein, wherein the anti-peptide agent preferentially binds to at least one of the covalently bound peptide tags over the non-analyte antibodies.

In a further embodiment, the system, kit, method, or detection complex of any one of the above embodiments and any one or more of the further embodiments herein, wherein the rate constant for binding of the anti-peptide agent to the tagged second agent is at least 2 times larger than the rate constants for binding of the binder to any of the non-analyte antibodies.

In a further embodiment, the system, kit, method, or detection complex of any one of the above embodiments and any one or more of the further embodiments herein, wherein the rate constant for binding of the anti-peptide agent to the tagged second agent is at least 5 times larger than the rate constants for binding of the binder to any of the non-analyte antibodies.

In a further embodiment, the system, kit, method, or detection complex of any one of the above embodiments and any one or more of the further embodiments herein, wherein the rate constant for binding of the anti-peptide agent to the tagged second agent is at least 10 times larger than the rate constants for binding of the binder to any of the non-analyte antibodies.

In a further embodiment, the system, kit, method, or detection complex of any one of the above embodiments and any one or more of the further embodiments herein, wherein the rate constant for binding of the anti-peptide agent to the tagged second agent is at least 100 times larger than the rate constants for binding of the binder to any of the non-analyte antibodies.

In a further embodiment, the system, kit, method, or detection complex of any one of the above embodiments and any one or more of the further embodiments herein, wherein the plurality of tagged first antibodies and/or the plurality of second antibodies are stored at 0-4° C.

In a further embodiment, the system, kit, method, or detection complex of any one of the above embodiments and any one or more of the further embodiments herein, wherein the plurality of tagged first agents and/or the plurality of second agents are stored at 0-4° C.

In a further embodiment, the system, kit, method, or detection complex of any one of the above embodiments and any one or more of the further embodiments herein, wherein the plurality of tagged first antibodies and/or the plurality of second antibodies are stored at room temperature.

In a further embodiment, the system, kit, method, or detection complex of any one of the above embodiments and any one or more of the further embodiments herein, wherein the plurality of tagged first agents and/or the plurality of second agents are stored at room temperature.

In a further embodiment, the system, kit, method, or detection complex of any one of the above embodiments and any one or more of the further embodiments herein, wherein the plurality of tagged first antibodies and/or the plurality of second antibodies are stored at less than 20° C.

In a further embodiment, the system, kit, method, or detection complex of any one of the above embodiments and any one or more of the further embodiments herein, wherein the plurality of tagged first antibodies and/or the plurality of second antibodies are stored at less than 30° C.

In a further embodiment, the system, kit, method, or detection complex of any one of the above embodiments and any one or more of the further embodiments herein, wherein the tagged first antibodies are in a first liquid buffer and the tagged second antibodies are in a second liquid buffer.

In a further embodiment, the system, kit, method, or detection complex of any one of the above embodiments and any one or more of the further embodiments herein, wherein the plurality of tagged first agents and/or the plurality of second agents are stored at less than 20° C.

In a further embodiment, the system, kit, method, or detection complex of any one of the above embodiments and any one or more of the further embodiments herein, wherein the plurality of tagged first agents and/or the plurality of second agents are stored at less than 30° C.

In a further embodiment, the system, kit, method, or detection complex of any one of the above embodiments and any one or more of the further embodiments herein, wherein the tagged first agents are in a first liquid buffer and the tagged second agents are in a second liquid buffer.

In a further embodiment, the system, kit, method, or detection complex of any one of the above embodiments and any one or more of the further embodiments herein, wherein the donor bead is stored under low light conditions.

In a further embodiment, the system, kit, method, or detection complex of any one of the above embodiments and any one or more of the further embodiments herein, wherein the detection system has a storage life of at least 9 months.

In a further embodiment, the system, kit, method, or detection complex of any one of the above embodiments and any one or more of the further embodiments herein, wherein the detection system has a storage life of at least one year.

In a further embodiment, the system, kit, method, or detection complex of any one of the above embodiments and any one or more of the further embodiments herein, wherein the first bead and the second bead are capable of interacting to produce a detectable emission.

In a further embodiment, the system, kit, method, or detection complex of any one of the above embodiments and any one or more of the further embodiments herein, wherein the first bead and the second bead are disposed within 200 nm of one another.

In a further embodiment, the system, kit, method, or detection complex of any one of the above embodiments and any one or more of the further embodiments herein, wherein the first bead and the second bead are disposed within 150 nm of one another.

In a further embodiment, the system, kit, method, or detection complex of any one of the above embodiments and any one or more of the further embodiments herein, wherein the first bead and the second bead are disposed within 50 nm of one another.

In a further embodiment, the system, kit, method, or detection complex of any one of the above embodiments and any one or more of the further embodiments herein, wherein the first bead and the second bead are disposed within 25 nm of one another.

In a further embodiment, the system, kit, method, or detection complex of any one of the above embodiments and any one or more of the further embodiments herein, wherein the one or more analyte complexes is between 1 and 5 analyte complexes.

In a further embodiment, the system, kit, method, or detection complex of any one of the above embodiments and any one or more of the further embodiments herein, wherein the one or more analyte complexes is between 2 and 4 analyte complexes.

In a further embodiment, the system, kit, method, or detection complex of any one of the above embodiments and any one or more of the further embodiments herein, wherein the donor antibody and the tagged further donor antibody are the same type of antibody.

In a further embodiment, the system, kit, method, or detection complex of any one of the above embodiments and any one or more of the further embodiments herein, wherein the donor agent and the tagged further donor agent are the same type of agent.

In a further embodiment, the system, kit, method, or detection complex of any one of the above embodiments and any one or more of the further embodiments herein, wherein the donor antibody and the tagged further donor antibody are different types of antibodies.

In a further embodiment, the system, kit, method, or detection complex of any one of the above embodiments and any one or more of the further embodiments herein, wherein the donor agent and the tagged further donor agent are different types of agents.

In a further embodiment, the system, kit, method, or detection complex of any one of the above embodiments and any one or more of the further embodiments herein, wherein the binder and the further binder are the same type of binder.

In a further embodiment, the system, kit, method, or detection complex of any one of the above embodiments and any one or more of the further embodiments herein, wherein:
i) the donor antibody and the tagged further donor antibody each comprise a plurality of biotin tags; and
ii) the binder and the further binder each comprise streptavidin.

In a further embodiment, the system, kit, method, or detection complex of any one of the above embodiments and any one or more of the further embodiments herein, wherein:
i) the donor antibody and the further donor antibody each comprise a plurality of peptide tags; and
ii) the binder and the further binder each comprise an anti-peptide agent conjugated to the donor bead.

In a further embodiment, the system, kit, method, or detection complex of any one of the above embodiments and any one or more of the further embodiments herein, wherein:
i) the donor antibody comprises a plurality of peptide second tags capable of binding with the binder, said binder comprising a second anti-peptide agent conjugated to the donor bead; and
ii) the further donor antibody comprises a plurality of peptide third tags capable of binding with the binder, said binder comprising a third anti-peptide agent conjugated to the donor bead.

In a further embodiment, the system, kit, method, or detection complex of any one of the above embodiments and any one or more of the further embodiments herein, wherein the further acceptor antibody is directly conjugated to the second acceptor bead.

In a further embodiment, the system, kit, method, or detection complex of any one of the above embodiments and any one or more of the further embodiments herein, wherein the further acceptor antibody comprises a plurality of covalently bound peptide second tags capable of binding to a second anti-peptide agent conjugated to the second acceptor.

In a further embodiment, the system, kit, method, or detection complex of any one of the above embodiments and any one or more of the further embodiments herein, wherein:
i) the donor agent and the tagged further donor agent each comprise a plurality of biotin tags; and
ii) the binder and the further binder each comprise streptavidin.

In a further embodiment, the system, kit, method, or detection complex of any one of the above embodiments and any one or more of the further embodiments herein, wherein:
i) the donor agent and the further donor agent each comprise a plurality of peptide tags; and
ii) the binder and the further binder each comprise an anti-peptide agent conjugated to the donor bead.

In a further embodiment, the system, kit, method, or detection complex of any one of the above embodiments and any one or more of the further embodiments herein, wherein:
i) the donor agent comprises a plurality of peptide second tags capable of binding with the binder, said binder comprising a second anti-peptide agent conjugated to the donor bead; and
ii) the further donor agent comprises a plurality of peptide third tags capable of binding with the binder, said binder comprising a third anti-peptide agent conjugated to the donor bead.

In a further embodiment, the system, kit, method, or detection complex of any one of the above embodiments and any one or more of the further embodiments herein, wherein the further acceptor agent is directly conjugated to the second acceptor bead.

In a further embodiment, the system, kit, method, or detection complex of any one of the above embodiments and any one or more of the further embodiments herein, wherein the further acceptor agent comprises a plurality of covalently bound peptide second tags capable of binding to a second anti-peptide agent conjugated to the second acceptor.

In a further embodiment, the method of any one of the above embodiments and any one or more of the further embodiments herein, wherein the method comprises:
i) forming a plurality of detection complexes dispersed in the sample, wherein the first beads are capable of a chemical transfer interactions with the second beads;
ii) irradiating the donor beads in at least a portion of the detection complexes with light at an appropriate wavelength to trigger excitation of at least a portion of the acceptor beads in the detection complexes; and
iii) detecting light emitted by the acceptor beads.

What is claimed is:

1. An analyte detection system, comprising:
i) a first agent comprising at least one tag and capable of binding to the analyte;
ii) a second agent covalently bound to a plurality of peptide tags and capable of binding to the analyte;
iii) a first bead comprising a binder capable of binding with the tagged first agent; and
iv) a second bead comprising an anti-peptide agent capable of binding with at least one of the plurality of covalently bound peptide tags.

2. The system of claim 1, wherein the anti-peptide agent comprises an affimer.

3. The system of claim 1, wherein the first agent comprises a first antibody.

4. The system of claim 3, wherein the at least one tag comprises a plurality of biotin or a derivative thereof.

5. The system of claim 4, wherein the first bead is coated with streptavidin or a derivative thereof.

6. The system of claim 1, wherein the second agent comprises a second antibody.

7. The system of claim 6, wherein the anti-peptide agent comprises an anti-peptide antibody.

8. The system of claim 7, wherein the anti-peptide antibody is present at a ratio of at least 100:1, on a weight:weight basis, relative to the second antibody.

9. The system of claim 1, wherein:
A) the first bead is a donor bead comprising a photosensitizer; and
B) the second bead is an acceptor bead configured to interact with the donor bead via a chemical transfer interaction.

10. The system of claim 9, wherein the donor bead emits singlet oxygen when stimulated by light at a wavelength in the range of between 650-700 nm.

11. The system of claim 9, wherein the acceptor bead comprises a photoactive indicator precursor that reacts with singlet oxygen forming a photoactive indicator, wherein irradiation of the photoactive indicator emits fluorescence at a wavelength in the range of between 500-625 nm.

12. The system of claim 9, wherein the first bead and the second bead each have a diameter in the range of 250-350 nm.

13. The system of claim 9, wherein the second bead is present at a ratio of in the range of 0.1-0.2, relative to the first bead.

14. An analyte detection kit, comprising:
i) a first agent comprising at least one tag and capable of binding to the analyte;
ii) a second agent covalently bound to a plurality of peptide tags and capable of binding to the analyte;
iii) a first bead comprising a binder capable of binding with the tagged first agent; and
iv) a second bead comprising an anti-peptide agent capable of binding with at least one of the plurality of covalently bound peptide tags.

15. A method for detecting an analyte, comprising:
i) binding an analyte with a first agent and a second agent, wherein:
a) the first agent comprises one or more first tags; and
b) the second agent comprises a plurality of covalently bound peptide second tags;
ii) attaching at least one of the one or more first tags to a binder conjugated to a first bead;
iii) binding at least one of the plurality of covalently bound peptide second tags to an anti-peptide agent conjugated to a second bead; and
iv) detecting the presence of the analyte by sensing an emission resulting from an interaction between the attached first bead and the bound second bead.

16. The method of claim 14, wherein the first epitope and the second epitope are the same type of epitope.

17. The method of claim 14, wherein the first epitope and the second epitope are two different types of epitopes.

18. A detection complex, comprising:
i) one or more analyte complexes, comprising:
a) an analyte;
b) a first agent comprising one or more first tags and bound to the analyte; and
c) a second agent covalently bound to a plurality of peptide tags and separately bound to said analyte;
ii) a first bead having a plurality of binders conjugated thereon, wherein at least one of the one or more first tags present from the one or more analyte complexes is bound to one of the plurality of binders; and
iii) a second bead having a plurality of anti-peptide antibodies conjugated thereon, wherein at least one of the plurality of covalently bound peptide second tags from the one or more analyte complexes is bound to one of the plurality of anti-peptide antibodies.

19. The detection complex of claim 18, wherein the first bead and the second bead are disposed within 50 nm of one another.

20. The detection complex of claim 18, wherein the one or more analyte complexes is between 1 and 5 analyte complexes.
